SEQUENCE LISTING (3 sequences)

SEQ ID NO: 1 - DNA, 219 bp, Pichia pastoris
gatgacgaaagctctaagccatgctgcgatcagtgtgcttgtaccaagtccaacccaccacagtgtagatgctccgacatgcgtttgaactcctgtcactccgcttgcaagtcctgtatctgtgccttgtcctacccagctcagtgtttctgcgtcgacatcaccgacttctgctacgagccatgtaagccatctgaggacgataaggaaaactaataa

SEQ ID NO: 2 - protein, 110 aa, Glycine max
MVVLKVCLVLLFLVGGTTSANLRLSKLGLLMKSDHQHSNDDESSKPCCDQCACTKSNPPQCRCSDMRLNSCHSACKSCICALSYPAQCFCVDITDFCYEPCKPSEDDKEN

SEQ ID NO: 3 - protein, 100 aa, Artificial Sequence
  Representative Sequence
MKMSRLCLSVALLVLLGTLAASTPGCDTSNQAKAQRPDFCLEPPYTGPCKARIIRYFYNAKAGLCQTFVYGGCRAKRNNFKSAEDCMRTCGGAIGPWENL

(19) United States
(12) Patent Application Publication
(10) Pub. No.: US 2021/0244806 A1
(43) Pub. Date: Aug. 12, 2021

(54) Recombinant Protease Inhibitor-Containing Compositions, Methods for Producing Same and Uses Thereof

(71) Applicant: ORAMED LTD., Jerusalem (IL)

(72) Inventors: Miriam Kidron, Jerusalem (IL); Kajal Arora, New Delhi (IN); Ramachandra Nagaraju, Haryana (IN); Nupur Mehrotra Arora, Haryana (IN); Prabuddha Kundu, Haryana (IN)

(21) Appl. No.: 16/973,929

(22) PCT Filed: Jun. 6, 2019
(86) PCT No.: PCT/IL2019/050647

§ 371 Date: Dec. 10, 2020

Related U.S. Application Data
(60) Provisional application No. 62/683,061, filed on Jun. 11, 2018.

(51) Int. Cl. A61K 38/56; A61K 36/06; A61K 38/28; A61K 47/18; A61K 45/06; A61K 47/14; A61K 47/42; A61K 47/10; A61K 47/44

(52) U.S. Cl. CPC A61K 38/56; A61K 36/06; A61K 38/28; A61K 47/183; A61K 47/44; A61K 47/14; A61K 47/42; A61K 47/10; A61K 45/06

(57) ABSTRACT

Provided herein are compositions for oral administration of therapeutic proteins and peptides, which compositions contain an isolated recombinantly expressed Bowman-Birk inhibitor (BBI) and which compositions provide for improved sustained activity of the therapeutic proteins and peptides. Methods of use of the compositions are provided, as well.

### FIELD

Provided herein are oral compositions for the administration of therapeutic proteins which contain a recombinant protease inhibitor exhibiting improved sustained activity.

### BACKGROUND

Protein/peptide-based drugs are typically susceptible to degradation in the gastrointestinal tract and/or are not efficiently absorbed into the bloodstream from the small intestine in bioactive form. Orally delivered formulations for protein-based drugs such as insulin, exenatide, other hormones, etc. have been developed.

Trypsin and chymotrypsin inhibitors derived from soybean (Glycine max) are readily available and are considered to be safe for human consumption. One example of such inhibitor has been termed SBTI (soybean trypsin inhibitor), which is composed of KTI (Kunitz Trypsin Inhibitor), which inhibits trypsin, and BBI (Bowman-Birk inhibitor), which inhibits trypsin and chymotrypsin. Various commercial sources for SBTI are known and improved methods for isolating the active components have been described in, for example, PCT International Application Publication Number WO/2013/114369, which is fully incorporated herein by reference in its entirety.

There has been tremendous progress and promise in terms of providing a platform technology for oral delivery of peptide-based therapeutics, in particular for therapeutics targeting metabolic conditions and given same, the ability to improve on same beyond the current state of the art would seem to be difficult to envision.

### SUMMARY

Surprisingly, it has now been shown that stable oral compositions comprising a peptide/protein-based therapeutic, an enhancer/stabilizer, an omega-3 fatty acid and protease inhibitor(s) already demonstrated to provide sustainable efficacy in an oral formulation, can be improved upon, when the protease inhibitor is derived from a recombinant source.

SBTI chemically extracted to obtain separate preparations of purified KTI and BBI, respectively, having known quantities of BBI and KTI, of known purity and activity when included in oral formulations has been used to prepare oral protein therapeutic-containing formulations which are effective in treating known diseases. For example, in PCT International Application Publication Number WO/2013/114369, which is herein incorporated by reference in its entirety, improved methods for the purification of SBTI were developed, in which each product was prepared under its own specifications to high levels of activity, and levels of high molecular weight (MW)-contaminants were minimized and industrial yield preparations of high purity were achieved.

Surprisingly, it has now been found, that when the protease inhibitors were prepared/derived from recombinant heterologous production means, despite shared downstream purification steps between chemically purified and recombinantly expressed proteases, the resulting therapeutic protein/peptide activity was greater and sustained over a prolonged period of time, when recombinant sources were used for the initial protease production source.

This invention provides an oral pharmaceutical composition comprising a therapeutic peptide or therapeutic protein of up to 100 kilodalton, a chelator of divalent cations, and an isolated recombinantly expressed BBI.

In some embodiments, the recombinantly expressed BBI has a nucleotide sequence sharing at least 95% identity with that set forth in SEQ ID NO: 1, or in some embodiments, the recombinantly expressed BBI has a nucleotide sequence sharing at least 97% identity with that set forth in SEQ ID NO: 1, or in some embodiments, the recombinantly expressed BBI has a nucleotide sequence sharing at least 99% identity with that set forth in SEQ ID NO: 1. In some embodiments, the recombinantly expressed BBI has a nucleotide sequence of that set forth in SEQ ID NO: 1.

In some embodiments, the recombinantly expressed BBI is expressed in a yeast expression system, which, in some embodiments, is in a Pichia pastoris yeast expression system.

In some embodiments, the recombinantly expressed BBI is expressed in a heterologous expression system, wherein the nucleotide sequence encoding same is optimized for codon usage in the organism being used as such heterologous expression system.

In some embodiments, the formulation further comprises a trypsin inhibitor other than said BBI, which in some embodiments, is KTI3, which in some embodiments, is chemically purified, and in some embodiments, is recombinantly expressed.

In some embodiments, the therapeutic protein is useful in treating a subject with a metabolic disease or disorder. In some embodiments, the therapeutic peptide or therapeutic protein is selected from the group consisting of insulin, influenza hemagglutinin, influenza neuraminidase, a glucagon, interferon gamma, interferon beta, interferon alpha, growth hormone, erythropoietin, GLP-1, a GLP-1 analogue, granulocyte colony stimulating factor (G-CSF), renin, growth hormone releasing factor, parathyroid hormone, thyroid stimulating hormone, follicle stimulating hormone, calcitonin, luteinizing hormone, glucagon, a clotting factor, an anti-clotting factor, atrial natriuretic factor, surfactant protein A (SP-A), surfactant protein B (SP-B), surfactant protein C (SP-C), surfactant protein D (SP-D), a plasminogen activator, bombesin, hemopoietic growth factor (colony-stimulating factor, multiple), a tumor necrosis factor (TNF) protein, enkephalinase, RANTES (regulated on activation normally T-cell expressed and secreted), human macrophage inflammatory protein (MIP-1-alpha), serum albumin, Mullerian-inhibiting substance, relaxin, mouse gonadotropin-releasing hormone, DNase, inhibin, activin, vascular endothelial growth factor (VEGF), a neurotrophic factor, neurotrophin-3,-4,-5, or -6 (NT-3, NT-4, NT-5, or NT-6), nerve growth factor, platelet-derived growth factor (PDGF), a fibroblast growth factor, a transforming growth factor (TGF), insulin-like growth factor-I and -II (IGF-I and IGF-II), des (1-3)-IGF-I (brain IGF-I), insulin-like growth factor binding protein 1 (IGFBP-1), IGFBP-2, IGFBP-3, IGFBP-4, IGFBP-5, IGFBP-6, a keratinocyte growth factor, an osteoinductive factor, bone morphogenetic protein (BMP)-2, BMP-7, a colony stimulating factor (CSF), an interleukin (IL), superoxide dismutase, decay accelerating factor, a chemokine family member, and a complement factor. In some embodiments, the therapeutic peptide or therapeutic protein is selected from the group consisting of insulin and a GLP-1 analogue. In some embodiments, the therapeutic peptide or therapeutic protein is selected from the group consisting of insulin and a GLP-1 analogue, Leptin, a bone morphogenetic protein including BMP-4 and nerve growth factor, including NGF-1.

In some embodiments, the therapeutic peptide or therapeutic protein is an immunomodulatory and in some embodiments, the therapeutic peptide or therapeutic protein is Glatiramer acetate. In some embodiments, the therapeutic peptide or therapeutic protein is provided to promote autotolerance to an allergen or antigen associated with autoimmune disease.

In some embodiments, the therapeutic peptide or therapeutic protein is useful in treating, mitigating, abrogating, reducing incidence or severity of a metabolic disease or disorder.

In some embodiments, this invention provides an oral pharmaceutical composition as herein described for use as a medicament for orally administering a therapeutic peptide or therapeutic protein to a subject.

In some aspect, the oral pharmaceutical compositions of this invention prevent rapid degradation of the therapeutic peptide or therapeutic protein in the subject, or in some embodiments, promote sustained activity of orally administered therapeutic peptides or therapeutic proteins in the subject, or in some embodiments, promote sustained bioavailability of orally administered therapeutic peptides or therapeutic proteins in the subject. According to this aspect and in some embodiments, the sustained activity or sustained bioavailability of orally administered therapeutic peptides or therapeutic proteins exceeds that obtained following the use of an otherwise identical oral formulation comprising a chemically purified BBI instead of said isolated recombinantly expressed BBI

In some embodiments the invention provides for the use of the oral pharmaceutical compositions as herein described for orally administering a therapeutic protein to a subject. In some embodiments, the oral pharmaceutical composition prevents rapid degradation of said therapeutic peptide or therapeutic protein in said subject. In some embodiments, the oral pharmaceutical composition promotes sustained activity of orally administered therapeutic peptides or therapeutic proteins in said subject. In some embodiments, the oral pharmaceutical composition promotes sustained bioavailability of orally administered therapeutic peptides or therapeutic proteins in said subject. In some embodiments, the use provides a therapeutically effective amount of said peptide or protein and wherein said BBI, or said therapeutic peptide or therapeutic protein of up to 100 kilodalton, or a combination thereof is provided in said formulation at a lower concentration than would be therapeutically effective in an otherwise identical oral formulation comprising a chemically purified BBI instead of said isolated recombinantly expressed BBI and said use achieves a comparable therapeutic effect in said subject.

In some embodiments, this invention provides a method for orally administering a therapeutic peptide or therapeutic protein to a subject, the method comprising the step of administering to a subject an oral pharmaceutical composition as herein described, thereby orally administering a therapeutic protein to a subject.

In some embodiments, this invention provides a method for of preventing degradation of orally administered therapeutic peptides or therapeutic proteins in a subject, said method comprising the step of administering to a subject an oral pharmaceutical composition as herein described.

In some embodiments, this invention provides a method of promoting sustained activity of orally administered therapeutic peptides or therapeutic proteins in a subject, said method comprising the step of administering to a subject an oral pharmaceutical composition as herein described.

In some embodiments, this invention provides a method of promoting sustained bioavailability of orally administered therapeutic peptides or therapeutic proteins in a subject, said method comprising the step of administering to a subject the oral pharmaceutical composition an oral pharmaceutical composition as herein described.

In some embodiments, this invention provides a method of providing a therapeutically effective amount of a peptide or protein in an oral formulation to a subject, said method comprising preparing an oral pharmaceutical composition comprising a therapeutic peptide or therapeutic protein of up to 100 kilodalton, a chelator of divalent cations, and an isolated recombinantly expressed Bowman-Birk inhibitor (BBI), wherein said BBI, or said therapeutic peptide or therapeutic protein of up to 100 kilodalton, or a combination thereof is provided in said formulation at a lower concentration than would be therapeutically effective in an otherwise identical oral formulation comprising a chemically purified BBI instead of said isolated recombinantly expressed BBI and achieving a comparable therapeutic effect in said subject.

### BRIEF DESCRIPTION OF THE DRAWINGS

The following figures are by way of illustrative example and are not meant to be taken as limiting the claimed invention.

FIG. 1. Chromatogram of purified rBBI. The result of the RPHPLC analysis is shown along with an overlay of same with an available standard.

FIG. 2. Protein Estimation By BCA. Lyophilized powder sample was weighed and dissolved in respective buffer (SOP No: QC-006) to make it at final concentration of 1 mg/ml for quantification. Results yielded Protein by BCA: 94%

FIG. 3. SDS-PAGE analysis of rBBI. Electrophoresis was performed, the gel was scanned, and bands were quantified using the Syngene gel documentation system in conjunction with Genesys as the software for capturing images Gene tool for densitometry quantification study. Samples of 1 milligram per milliliter (mg./ml.) were loaded on a 20% Phastgel™. Lane 1: Protein Marker, Lane 2, blank, Lane 3, rBBI 2 μg under reducing conditions, Lane 4, rBBI 5 μg under reducing conditions, Lane 5, rBBI 10 μg under reducing conditions, Lane 6 Blank, Lane 7, rBBI 2 μg under non-reducing conditions, Lane 4, rBBI 5 μg under non-reducing conditions, Lane 5, rBBI 10 μg under non-reducing conditions.

FIG. 4. Chymotrypsin Inhibition Assay. Results demonstrated Sample showing 2.3 mg chymotrypsin inhibited by 1 mg inhibitor

FIG. 5. Trypsin Inhibition Assay. Results demonstrated Sample showing 1.7 mg trypsin inhibited by 1 mg inhibitor

FIG. 6. Average blood glucose profiles following administration of oral insulin formulations over time. In vivo study conducted in fasting pigs treated with oral insulin formulations (8 mg) prepared with a protease inhibitor (BBI) derived from natural sources or engineered using recombinant expression technology. Formulations were inserted to the duodenum using endoscopy, to final doses of 4 mg insulin. Blood samples were drawn throughout the 5-hour monitoring period for determination of glucose concentrations. In addition to 8 mg insulin and 150 mg EDTA, each formulation contained, as indicated, SBTI as described in WO 2013/114369, or recombinant BBI (rBBI) 100 mg, rBBI 100 mg and 25 mg KTI or 50 mg rBBI and 25 mg KTI. Each group administered an rBBI containing composition experienced a more precipitous drop in circulating glucose levels as compared to animals receiving chemically purified BBI from SBTI starting materials.

### DETAILED DESCRIPTION OF THE EMBODIMENTS

This invention provides improved oral compositions containing one or more therapeutic peptides or proteins, wherein a recombinant protease inhibitor, BBI is included in same, which surprisingly outperforms compositions containing the same protease inhibitor purified chemically, this despite comparable downstream purification steps for each. Surprisingly, it has now been shown that stable oral compositions comprising a peptide/protein-based therapeutic, an enhancer/stabilizer, an omega-3 fatty acid and protease inhibitor(s) already demonstrated to provide sustainable efficacy in an oral formulation, can be improved upon, when the protease inhibitor is derived from a recombinant source.

This invention provides an oral pharmaceutical composition comprising an oil-based liquid formulation, wherein said oil-based liquid formulation comprises a therapeutic peptide or therapeutic protein of up to 100 kilodalton, a chelator of divalent cations, and an isolated recombinantly expressed BBI.

In some embodiments, the recombinantly expressed BBI has a nucleotide sequence sharing at least 95% identity with that set forth in SEQ ID NO: 1, or in some embodiments, the recombinantly expressed BBI has a nucleotide sequence sharing at least 97% identity with that set forth in SEQ ID NO: 1, or in some embodiments, the recombinantly expressed BBI has a nucleotide sequence sharing at least 99% identity with that set forth in SEQ ID NO: 1. In some embodiments, the recombinantly expressed BBI

The terms “protein” and “peptide” are used interchangeably herein. Neither term is intended to confer a limitation of the number of amino acids present, except where a limitation is explicitly indicated.

In some embodiments, the oral formulations as described herein contain recombinantly expressed BBI, and in some embodiments, may further comprise additional protease inhibitors, such as, for example, chemically purified protease inhibitors, for example, such as KTI3 isolated from soybean flour, from traditional commercially available SBTI preparations, as described.

As exemplified herein in Example 4, oral formulations containing the subclinical 4 mg insulin dose provided for superior glycemic control, in terms of rapid circulating glucose levels, which effects were sustained for more than at least 3 hours, when 25 mg rBBI was included in the formulation. This effect was 75% longer than the duration of the effect measured in the control animals. Similarly, when 8 mg insulin was administered, the rBBI containing formulations outperformed formulations containing chemically extracted SBTI.

In terms of the ratio of the anti-trypsin activity to the anti-chymotrypsin activity present in the rBBI containing preparations, the activity ranged from between 1.6:1 and 1:1 inclusive. In terms of the ratio of the anti-trypsin activity to the anti-chymotrypsin activity present in the rBBI containing preparations, the activity was also demonstrated to range from between 1.5:1 and 1:1 inclusive.

Unless indicated otherwise, anti-chymotrypsin activity referred to herein is measured using chymotrypsin having an activity of 40 BTEE units per mg. of chymotrypsin, and is expressed in mg. of chymotrypsin inhibited per mg. of protein being tested. BTEE refers to N-Benzoyl-L-Tyrosine Ethyl Ester (see the directions for Sigma-Aldrich Product No. B6125).

Unless indicated otherwise, anti-trypsin activity referred to herein is measured using trypsin having an activity of 10,000 BAEE units per mg. of trypsin, and is expressed in mg. of trypsin inhibited per mg. of protein being tested. BAEE refers to Na-Benzoyl-L-Arginine Ethyl Ester Solution (see the directions for Sigma-Aldrich Product No. B4500). For example, in a typical assay, one unit corresponds to the amount of inhibitor that reduces the trypsin activity by one benzoyl-L-arginine ethyl ester unit (BAEE-U). One BAEE-U is the amount of enzyme that increases the absorbance at 253 nm by 0.001 per minute at pH 7.6 and 25° C. See, for example, K. Ozawa, M. Laskowski, 1966, J. Biol. Chem. 241:3955; and Y. Birk, 1976, Meth. Enzymol. 45:700.

In an additional aspect, a KTI3 isolated from soy flour is provided, wherein the KTI3 is at least 85% pure as measured, in various embodiments, by SDS-PAGE, Brilliant Blue staining, or imager quantitation.

In yet another aspect is provided a KTI3 isolated from soy flour, wherein the protein content of the KTI3 is greater than 95% as measured by BCA assay.

In yet another aspect is provided a KTI3 isolated from soy flour, wherein the KTI3 contains less than 0.1% high-MW contaminants, for example as assessed by SDS-PAGE and imager quantitation.

Those skilled in the art will appreciate that each of the above purity requirements, regarding its protein content, level of contaminants, or potency, is typically assessed prior to the KTI3 being mixed with one or more other components of the pharmaceutical composition.

In further embodiments, the above-described pharmaceutical compositions comprise a coating that resists degradation in the stomach. In even more specific embodiments, the coating is a pH-sensitive capsule, or alternatively, is a soft gelatin capsule.

In other embodiments, the above-described pharmaceutical compositions further comprise a therapeutic protein of up to 100 kilodaltons as an active ingredient. In other embodiments, the active ingredient is a non-protein molecule that is sensitive to degradation or inactivation in the human digestive tract.

In another embodiment, an oral pharmaceutical composition is provided, comprising an oil-based liquid formulation, wherein the oil-based liquid formulation comprises a therapeutic protein of up to 100 kilodaltons (kDa), a chelator of divalent cations, and a recombinant BBI as herein described, and optionally additional protease inhibitors. In other embodiments, the liquid formulation consists essentially of a therapeutic protein of up to 100 kDa, a chelator of divalent cations, a recombinant BBI as herein described, and an oil. In other embodiments, the liquid formulation consists essentially of a therapeutic protein of up to 100 kDa, a chelator of divalent cations, a recombinant BBI as herein described, an oil, and an emulsifier. In other embodiments, the liquid formulation consists essentially of a therapeutic protein of up to 100 kDa, a chelator of divalent cations, a recombinant BBI as herein described, an oil, and two emulsifiers.

In another aspect is provided an oral pharmaceutical composition comprising an oil-based liquid formulation, wherein the oil-based liquid formulation comprises a therapeutic protein of up to 100 kDa and a chelator of divalent cations, and a recombinant BBI as herein described such that said liquid formulation has an anti-chymotrypsin activity of at least 50 mg chymotrypsin inhibited per ml. of the liquid formulation. In other embodiments, the liquid formulation has an anti-chymotrypsin activity of at least 35, 40, 45, 55 or 60 mg. chymotrypsin inhibited per ml. of the liquid formulation. In still other embodiments, the liquid formulation has an anti-chymotrypsin activity in the range of 35-70, 40-70, 45-70, 50-70, or 40-60 mg. of chymotrypsin inhibited per ml. of the liquid formulation. In other embodiments, the liquid formulation further comprises an anti-trypsin activity of at least 25 mg. of trypsin inhibited per ml. of the liquid formulation. In other embodiments, the liquid formulation further comprises an anti-trypsin activity of at least 30, 35, 40, 45, or 50 mg. trypsin inhibited per ml. of the liquid formulation. Alternatively, the liquid formulation further comprises an anti-trypsin activity in the range of 25-50, 30-50, 35-50, 25-40, or 25-45 mg. trypsin inhibited per ml. of the liquid formulation.

In another aspect is provided a method for making a pharmaceutical composition, comprising the steps of (a) providing a preparation of a recombinant BBI as herein described, a therapeutic protein of up to 100 kilodaltons, and a chelator of divalent cations; and (b) mixing said recombinant BBI as herein described, therapeutic protein, and chelator into an oil-based liquid formulation. In addition, each of the embodiments described herein of the other ingredients, and of additional ingredients that may be present, may be incorporated into this method. In other embodiments, a pharmaceutical composition made by this method is provided.

“Liquid” as used herein refers to a composition that has a viscosity within the range of 1-1000 millipascal seconds, inclusive, at 20° C. Fish oil, for instance, is a liquid under ambient conditions. The term includes oil-based solutions, suspensions, and combinations thereof.

In certain embodiments, BBI refers to Bowman-Birk inhibitor; Uniprot number P01055 [database accessed on Jan. 28, 2013]).

A representative precursor sequence of BBI is:

(SEQ ID NO: 2)
MVVLKVCLVL LFLVGGTTSA NLRLSKLGLL MKSDHQHSND
DESSKPCCDQ CACTKSNPPQ CRCSDMRLNS CHSACKSCIC
ALSYPAQCFC VDITDFCYEP CKPSEDDKEN.

In some embodiments, KTI as used herein refers to KTI3 (Uniprot number P01070; database accessed on Jan. 3, 2013). A representative precursor sequence of KTI3 is:

SEQ ID NO: 2)
MKSTIFFLFL FCAFTTSYLP SAIADFVLDN EGNPLENGGT
YYILSDITAF GGIRAAPTGN ERCPLTVVQS RNELDKGIGT
IISSPYRIRF IAEGHPLSLK FDSFAVIMLC VGIPTEWSVV
EDLPEGPAVK IGENKDAMDG WFRLERVSDD EFNNYKLVFC
PQQAEDDKCG DIGISIDHDD GTRRLVVSKN KPLVVQFQKL
DKESLAKKNH GLSRSE

In some aspects, KTI3 may be included in the oral compositions as herein described as chemically purified, for example as available commercially, as purified from soybean flour.

In other aspects, the KTI3 may be recombinantly produced, as well.

Additional Protease Inhibitors

Some trypsin inhibitors known in the art are specific to trypsin, while others inhibit trypsin and other proteases such as chymotrypsin. Trypsin inhibitors can be derived from animal or vegetable sources: for example, soybean, corn, lima and other beans, squash, sunflower, bovine and other animal pancreas and lung, chicken and turkey egg white, soy-based infant formula, and mammalian blood. Trypsin inhibitors can also be of microbial origin: for example, antipain; see, for example, H. Umezawa, 1976, Meth. Enzymol. 45, 678. A trypsin inhibitor can also be an arginine or lysine mimic or other synthetic compound: for example arylguanidine, benzamidine, 3,4-dichloroisocoumarin, diisopropylfluorophosphate, gabexate mesylate, or phenylmethanesulfonyl fluoride. As used herein, an arginine or lysine mimic is a compound that is capable of binding to the P¹ pocket of trypsin and/or interfering with trypsin active site function.

In certain embodiments, the additional trypsin inhibitor utilized in the described methods and compositions is selected from the group consisting of lima bean trypsin inhibitor, aprotinin, (a.k.a. pancreatic trypsin inhibitor or basic pancreatic trypsin inhibitor [BPTI]; Uniprot No. P00974 [database accessed on Jan. 2, 2013]), Kazal inhibitor (pancreatic secretory trypsin inhibitor), Kazal inhibitor (pancreatic secretory trypsin inhibitor), ovomucoid, Alpha 1-antitrypsin, Cortisol binding globulin, Centerin ([SERPINA9/GCET1 (germinal centre B-cell-expressed transcript 1)], PI-6 (Sun et al 1995), PI-8 (Sprecher et al 1995), Bomapin, a Glade A serpin [for example Serpina3 (NCBI Gene ID: 12), Serpina6 (NCBI Gene ID: 866), Serpina12 (NCBI Gene ID: 145264); Serpina10 (NCBI Gene ID: 51156); Serpina7 (NCBI Gene ID: 6906); Serpina9 (NCBI Gene ID: 327657); Serpina11 (NCBI Gene ID: 256394); Serpina13 (NCBI Gene ID: 388007); Serpina2 (NCBI Gene ID: 390502); and Serpina4 (NCBI Gene ID: 5104)] Yukopin (Serpinb12; Gene ID: 89777), antipain, benzamidine, 3,4-dichloroisocoumarin, diisopropylfluorophosphate, and gabexate mesylate. In other embodiments, one of the above inhibitors is selected.

A representative precursor sequence of aprotinin is:

(SEQ ID NO: 3)
MKMSRLCLSV ALLVLLGTLA ASTPGCDTSN QAKAQRPDFC
LEPPYTGPCK ARIIRYFYNA KAGLCQTFVY GGCRAKRNNF
KSAEDCMRTC GGAIGPWENL.

In other embodiments, an oil-based liquid formulation utilized in the described methods and compositions comprises both recombinant BBI and isolated or recombinant KTI, or isolated and recombinant aprotinin, or a combination thereof.

In certain embodiments, the rBBI utilized in the described methods and compositions, and optional second protease inhibitor such as KTI, if present, has been stored with a preservative. In other embodiments, the rBBI and optional second protease inhibitor such as

KTI has been prepared and stored without use of a preservative.

Therapeutic Proteins

Therapeutic proteins for compositions and methods described herein are in some embodiments isolated prior to inclusion in the described pharmaceutical compositions. “Isolated” in this regard excludes provision of the therapeutic protein as a homogenized tissue preparation or other form containing substantial amounts of contaminating proteins. A preferred example of an isolated protein or peptide is a recombinant protein or peptide. An even more preferred embodiment is a synthetic protein, in other words a protein produced in a cell-free apparatus. Those skilled in the art will appreciate in light of the present disclosure that both wild-type and mutated therapeutic proteins may be utilized.

Certain proteins and peptides are known to be specific inhibitors of trypsin and/or chymotrypsin, including but not limited to those described herein as being trypsin and/or chymotrypsin inhibitors. Such proteins are not intended for use as the therapeutic component in the described compositions, and are excluded from the definition of “therapeutic proteins” as used herein.

Those of skill in the art will appreciate in light of the present disclosure that a variety of therapeutic proteins may be used in the described methods and compositions. In certain embodiments, the therapeutic protein is up to 100 kilodaltons (kDa) in size, typically between 1-100 kDa, inclusive. In more specific embodiments, the size is up to 90 kDa. In other embodiments, the size is up to 80 kDa. In other embodiments, the size is up to 70 kDa. In other embodiments, the size is up to 60 kDa. In other embodiments, the size is up to 50 kDa. Preferably, the size is between 1-90 kDa, inclusive. In other embodiments, the size is between 1-80 kDa, inclusive. In other embodiments, the size is between 1-70 kDa, inclusive. In other embodiments, the size is between 1-60 kDa, inclusive. In other embodiments, the size is between 1-50 kDa, inclusive.

Therapeutic proteins suitable for use herein include derivatives that are modified (i.e., by the covalent attachment of a non-amino acid moiety to the protein). For example, but not by way of limitation, the protein includes proteins that have been modified, e.g., by glycosylation, acetylation, PEGylation, phosphorylation, amidation, genetically added polypeptides as fusion proteins or derivatization by known protecting/blocking groups. High-MW PEG can be attached to therapeutic proteins with or without a multifunctional linker either through site-specific conjugation of the PEG to the N- or C-terminus thereof or via epsilon-amino groups present on lysine residues. Additionally, the derivative may contain one or more non-classical amino acids.

In certain, more specific, embodiments, the therapeutic protein utilized in the described methods and compositions is selected from the group consisting of insulin, influenza hemagglutinin, influenza neuraminidase, glucagon, interferon gamma, interferon beta, interferon alpha, growth hormone, erythropoietin, GLP-1, a GLP-1 analogue, leptin, granulocyte colony stimulating factor (G-CSF), renin, growth hormone releasing factor, parathyroid hormone, thyroid stimulating hormone, follicle stimulating hormone, calcitonin, luteinizing hormone, glucagon, a clotting factor (for example factor VII, factor VIIIC, factor DC, tissue factor (TF), and thrombin), an anti-clotting factor (for example Protein C), atrial natriuretic factor, surfactant protein A (SP-A), surfactant protein B (SP-B), surfactant protein C (SP-C), surfactant protein D (SP-D), a plasminogen activator (for example urokinase or human urine or tissue-type plasminogen activator (t-PA)), bombesin, hemopoietic growth factor (a.k.a. colony-stimulating factor, multiple), a tumor necrosis factor (TNF) protein (for example TNF-alpha, TNF-beta, TNF beta-2, 4-1BBL), enkephalinase, RANTES (regulated on activation normally T-cell expressed and secreted), human macrophage inflammatory protein (MIP-1-alpha), serum albumin, Mullerian-inhibiting substance, relaxin, mouse gonadotropin-releasing hormone, DNase, inhibin, activin, vascular endothelial growth factor (VEGF), a neurotrophic factor (for example brain-derived neurotrophic factor [BDNF]), neurotrophin-3, -4, -5, or -6 (NT-3, NT-4, NT-5, or NT-6), nerve growth factor, platelet-derived growth factor (PDGF), a fibroblast growth factor (for example alpha-FGF and beta-FGF), a transforming growth factor (TGF) (for example TGF-alpha and TGF-beta, including TGF-1, TGF-2, TGF-3, TGF-4, and TGF-5), a nerve growth factor including NGF-1, insulin-like growth factor-I and -II (IGF-I and IGF-II), des (1-3)-IGF-I (brain IGF-I), insulin-like growth factor binding proteins (including IGFBP-1, IGFBP-2, IGFBP-3, IGFBP-4, IGFBP-5, and IGFBP-6), a keratinocyte growth factor, an osteoinductive factor, bone morphogenetic protein (BMP)-2, BMP-7, BMP-4, a colony stimulating factor (CSF) (for example M-CSF and GM-CSF), an interleukin (IL), (for example IL-1 to IL-13 and IL-15, IL-18, and IL-23), superoxide dismutase, decay accelerating factor, a chemokine family member (for example the eotaxins and MCP-1), and a complement factor (for example C3 and C5).

In still other aspects, the therapeutic protein/peptides for incorporation in the oral formulations of this invention include therapeutic peptides/proteins which function as enzyme replacement therapy (ERT) in patients afflicted by, e.g. a hereditary metabolic diseases, in which genetic defects result in the structural defect or absence of endogenous enzymes critical for metabolic pathways. These metabolic defects in turn, result in the accumulation of substrates and/or its intermediates, leading to pathological states. For example, and in some embodiments, the therapeutic proteins/peptides may include Glucocerebrosidase, α-galactosidase, Acid α-glucosidase, α-L-iduronidase, Iduronate-2-sulfatase, N-acetylgalactosamine4-sulfatase, Arylsulfatase A, Acid sphingomyelinase, alkaline phosphatase, pyridoxal 5-phosphate, Porphobilinogen deaminase and Phenylalanine ammonia lyase.

In other embodiments, the therapeutic proteins/peptides serve to alleviate symptoms of pathological conditions by augmenting existing endogenous pathways, or by exerting novel catalytic functions. Such therapeutic enzymes have been successfully utilized in a variety of pathological conditions ranging from oncology, to neuromuscular dysfunctions, to hemostasis, and even cosmetic surgery. For example, and in some embodiments, the therapeutic proteins/peptides may include

Alteplase, Reteplase, Tenecteplase, human tissue plasminogen activator, Urokinase, Streptokinase, Anistreplace Eminases, Drotrecognin-a, Factor VIIa, human coagulation Factor VIIa, L-Asparaginase, Carboxypeptidase Rasburicase, Adenosine deaminase, Botulism toxin type A, Hyaluronidase and others.

In other embodiments, the therapeutic proteins/peptides may comprise Factor VIIa, Factor VIII, Factor IX, Factor X, Factor XI, Factor XIII, vWF, Protein C, Antithrombin III, Fibrinogen, C1-esterase inhibitor, Alpha-1 proteinase inhibitor (α1-PI), Glucocerebrosidase, Alpha-L-iduronidase, Iduronate sulfatase N-acetylgalactosamine-4-sulfatase, N-acetylgalactosamine-6-sulfatase, Heparan sulfate sulfatase, Alpha-galactosidase A, Alpha-glucosidase, Acid sphingomyelinase, Alpha-mannosidase, Arylsulphatase A, Lysosomal acid lipase (LAL), Sucrase-isomaltase, Adenosine deaminase Insulin-like growth factor 1 (IGF-1), Alkaline phosphatase, Porphobilinogen deaminase or combinations thereof.

In still more specific embodiments, the therapeutic protein is insulin. Alternatively, the therapeutic protein may be a GLP-1 inhibitor. In a more specific embodiment, the therapeutic protein is exenatide. In other embodiments, both insulin and exenatide are present in the described composition. In other embodiments, the liquid formulation consists essentially of insulin, exenatide, a chelator of divalent cations, an isolated BBI, and an oil. In other embodiments, the liquid formulation consists essentially of insulin, exenatide, a chelator of divalent cations, an isolated BBI, at least one emulsifier, and an oil. In still other embodiments, the liquid formulation consists essentially of insulin, exenatide, a chelator of divalent cations, an isolated KTI3, aprotinin, and an oil. In yet embodiments, the liquid formulation consists essentially of insulin, exenatide, a chelator of divalent cations, an isolated KTI3, aprotinin, at least one emulsifier, and an oil.

A person skilled in the art will appreciate in light of the present disclosure that various types of insulin are suitable for the described methods and compositions. Exemplary insulin proteins include but are not limited to both wild-type and mutated insulin proteins, including synthetic human insulin, synthetic bovine insulin, synthetic porcine insulin, synthetic whale insulin, and metal complexes of insulin, such as zinc complexes of insulin, protamine zinc insulin, and globin zinc.

Various classes of insulin may also be utilized, for example fast-acting insulin, lente insulin, semilente insulin, ultralente insulin, NPH insulin, glargine insulin, lispro insulin, aspart insulin, or combinations of two or more of the above types of insulin.

In a particularly preferred embodiment, the insulin of the described methods and compositions is wild-type human insulin (Uniprot ID P01308). Of the 110 amino acids, 1-24 is the signal peptide, 25-54 forms the insulin B chain, 57-87 forms C peptide, and 90-110 forms the insulin A chain. In one preferred embodiment, human insulin is produced as a recombinant protein in bacterial cells. In another preferred embodiment, human insulin is produced synthetically.

GLP-1 analogues are also referred to in the art as GLP-1 mimetics. A person of skill in the art will appreciate in light of the present disclosure that the described compositions may include at least one of the following GLP-1 analogues: exenatide (Byetta™; CAS no. 141732-76-5), lixisenatide (CAS no. 320367-13-3), liraglutide (CAS no. 204656-20-2), exendin-9 (CAS no. 133514-43-9), AC3174 ([Leu(14)]exendin-4, Amylin Pharmaceuticals, Inc.), taspoglutide (CAS no. 275371-94-3), albiglutide (CAS no. 782500-75-8), semaglutide (CAS no. 910463-68-2), LY2189265 (dulaglutide™; CAS no. 923950-08-7), and CJC-1134-PC (a modified Exendin-4 analogue conjugated to recombinant human albumin manufactured by ConjuChem™). All CAS records were accessed on Dec. 19, 2011. Thus, in certain embodiments, the described method or composition utilizes any of the above-listed GLP-1 analogues. In other embodiments, one of the above-listed GLP-1 analogues is selected. Those of skill in the art will appreciate in light of the findings presented herein that other GLP-1 analogues can also be utilized in the described methods and compositions.

In some embodiments, reference to the “therapeutic peptide” or “therapeutic protein” is intended to include any such molecule, which when provided to a subject in need, provides a beneficial effect. In some cases, the molecule is therapeutic in that it functions to replace an absence or diminished presence of such a molecule in a subject. In one embodiment, the molecule substitutes for a protein that is absent, such as in cases of an endogenous null mutant being compensated for by oral delivery of the foreign protein. In other embodiments, the endogenous protein is mutated, and produces a non-functional protein, compensated for by the oral delivery of a heterologous functional protein. In other embodiments, oral delivery of a heterologous protein is additive to low endogenous levels, resulting in cumulative enhanced availability of a given protein. In other embodiments, the molecule stimulates a signaling cascade that provides for expression, or secretion, or others of a critical element for cellular or host functioning.

Emulsifiers

“Weight/weight” percentages of emulsifiers and detergents referred to herein utilize the amount of oil base in the formulation, for example fish oil, as the denominator; thus, 60 mg of Gelucire in 500 mg fish oil is considered as 12% w/w, regardless of the weight of the other components. Similarly, 50 mg. Tween-80 mixed with 500 mg fish oil is considered as 10% Tween-80.

In certain embodiments, the oil-based liquid formulation utilized in the described methods and pharmaceutical compositions, or in other embodiments, each of the oil-based liquid formulation that is present, comprises, in addition to the therapeutic protein, chelator, and BBI, a polyethylene glycol (PEG) ester of a fatty acid, for example a PEG ester of a monoglyceride, a diglyceride, a triglyceride, or a mixture thereof. In more specific embodiments, the PEG ester may be provided as a mixture of (a) a free monoacylglycerol, a free diacylglycerol, a free triacylglycerol, or a mixture thereof; and (b) a PEG ester of a fatty acid, for example a PEG ester of a monoglyceride, a diglyceride, a triglyceride, or a mixture thereof. In this regard, each of the terms “monoacylglycerol”, “diacylglycerol”, and “triacylglycerol” need not refer to a single compound, but rather can include mixtures of compounds, for example mixtures of monoacylglycerols, diacylglycerols, or triacylglycerols having fatty acids of varying lengths. In certain preferred embodiments, monoacylglycerols, diacylglycerols, or triacylglycerols utilized in the described methods and compositions, for example those used to general PEG esters, are from an oil source that is Generally Recognized As Safe (GRAS). Examples of GRAS oils are coconut oil, corn oil, peanut oil, soybean oil, Myvacet 9-45 (Diacetylated monoacylglycerols of C-18 fatty acids). A more specific embodiment of (a) is a mixture of C₈-C₁₈ monoacylglycerols, diacylglycerols, and triacylglycerols. A more specific embodiment of component (b) is a mixture of PEG monoesters and diesters of one or more C₈-C₁₈ fatty acids.

In more specific embodiments, the liquid formulation further comprises, in addition to the PEG ester of a fatty acid, a free PEG. In still more specific embodiments, an additional non-ionic detergent, for example a polysorbate-based detergent, is present in addition to the PEG ester and free PEG.

In a still more specific embodiment of the described methods and compositions, a liquid formulation comprises: (a) a mixture of C₈-C₁₈ monoacylglycerols, diacylglycerols, and triacylglycerols; (b) PEG-32 monoesters and diesters of a mixture of C₈-C₁₈ fatty acids; and (c) free PEG-32. In even more specific embodiments, the weight/weight ratio of component (a) to the sum of components (b)+(c) is between 10:90-30:70 inclusive; more specifically between 15:85-25:75 inclusive; more specifically 20:80. In certain embodiments, components (a)-(c) together constitute 8-16% weight/weight inclusive of the oil-based liquid formulation. In more specific embodiments, the amount is 9-15% inclusive. In more specific embodiments, the amount is 10-14% inclusive. In more specific embodiments, the amount is 11-13% inclusive. In more specific embodiments, the amount is 12%.

In other embodiments, an oil-based liquid formulation utilized in the described methods and pharmaceutical compositions comprises, in addition to the therapeutic protein, chelator, and BBI, a self-emulsifying component. While some embodiments of self-emulsifying components are the mixtures of components described in the preceding paragraphs, these mixtures do not limit the definition of the term “self-emulsifying components” as used herein. “Self-emulsifying component” as used herein refers to a component that spontaneously forms an emulsion. Typically, such components will form an emulsion on contact with aqueous media, forming a fine dispersion i.e. a microemulsion (SMEDDS). Certain embodiments of such components comprise a triacylglycerol mixture of triacylglycerols and a high hydrophile/lipophile balance (HLB; see Griffin WC: “Calculation of HLB Values of Non-Ionic Surfactants,” J Soc Cosmetic Chemists 5:259 (1954)) surfactant. Other embodiments of the self-emulsifying component have a waxy, semi solid consistency.

Preferably, the HLB of a self-emulsifying component utilized in the described methods and compositions is 10 or greater. In other embodiments, it is between 11-19, inclusive. In other embodiments, it is between 12-18, inclusive. In other embodiments, it is between 12-17, inclusive. In other embodiments, it is between 12-16, inclusive, which is indicative of an oil-in-water (O/W) emulsifier. In other embodiments, it is between 13-15, inclusive. In other embodiments, it is 14. Still more specific embodiments of self-emulsifying components have an HLB of 12-16 inclusive and comprise medium- and long-chain triacylglycerols conjugated to PEG, free triacylglycerols, and free PEG. In other embodiments, the self-emulsifying component has an HLB of 12-16 inclusive and consists of a mixture of medium- and long-chain triacylglycerols conjugated to PEG, free triacylglycerols, and free PEG. In other embodiments, the self-emulsifying component has an HLB of 14 and comprises medium- and long-chain triacylglycerols conjugated to PEG, free triacylglycerols, and free PEG. In other embodiments, the self-emulsifying component has an HLB of 14 and consists of a mixture of medium- and long-chain triacylglycerols conjugated to PEG, free triacylglycerols, and free PEG.

Certain, more specific embodiments utilize self-emulsifying components that comprise (a) a monoacylglycerol, a diacylglycerol, a triacylglycerol, or a mixture thereof; and (b) a polyethylene glycol (PEG) ester of a fatty acid. In this regard, each of the terms “monoacylglycerol”, “diacylglycerol”, and “triacylglycerol” need not refer to a single compound, but rather can include mixtures of compounds, for example mixtures of monoacylglycerols, diacylglycerols, or triacylglycerols having fatty acids of varying lengths. A more specific embodiment is a mixture of C₈-C₁₈ monoacylglycerols, diacylglycerols, and triacylglycerols. A more specific embodiment of component (b) is a mixture of PEG monoesters and diesters of a mixture of C₈-C₁₈ fatty acids.

In other, more specific embodiments, the self-emulsifying component further comprises free PEG.

Certain PEG moieties for use in the described compositions and methods contain between 5-100 monomers. In more specific embodiments, the PEG may contain between 15-50 monomers. In still more specific embodiments, the PEG may contain between 25-40 monomers. In more specific embodiments, the PEG may contain 32 monomers.

In still more specific embodiments of the described methods and compositions, a self-emulsifying component used therein comprises: (a) a mixture of C₈-C₁₈ monoacylglycerols, diacylglycerols, and triacylglycerols; (b) PEG-32 monoesters and diesters of a mixture of C₈-C₁₈ fatty acids; and (c) free PEG-32; and the weight/weight ratio of component (a) to components (b)+(c) is 20:80. In certain embodiments, such a component constitutes 8-16% weight/weight inclusive of the oil-based liquid formulation. In more specific embodiments, the amount is 9-15% inclusive. In more specific embodiments, the amount is 10-14% inclusive. In more specific embodiments, the amount is 11-13% inclusive. In more specific embodiments, the amount is 12%.

Examples of self-emulsifying components meeting the above specifications are Gelucire™ 44/14, Gelucire™ 53/10, and Gelucire™ 50/13. A more specific embodiment is Gelucire™ 44/14. The suffixes 44 and 14 refer respectively to its melting point and its hydrophilic/lypophilic balance (HLB). Gelucire™ 44/14 (Gattefossé SAS, Saint-Priest, France) is obtained by polyglycolysis of hydrogenated coconut oil (medium- and long-chain triacylglycerols with PEG-32. It has a hydrophile/lipophile balance of 14. It is composed of a defined admixture of C₈-C₁₈ mono-, di- and triacylglycerols (20% w/w); PEG-32 mono- and diesters and free PEG-32 (80% w/w). The main fatty acid present is lauric acid, accounting for 45% on average of the total fatty acid content. It is a solid dispersion composed of a PEG ester fraction under a lamellar phase of 120 Å with a helical conformation and an acylglycerol fraction under a hexagonal packing. The main products of simulated gastrointestinal lipolysis of Gelucire™ 44/14 are PEG-32 mono and diesters. In more specific embodiments, the described compositions comprise about 12% Gelucire 44/14 as the only emulsifier, or, in other embodiments, together with another emulsifier. In other embodiments, the described compositions comprise about 12% Gelucire 44/14 and about 10% Tween-80.

Non-Ionic Detergents

In certain embodiments, an oil-based liquid formulation utilized in the described methods and pharmaceutical compositions further comprises a non-ionic detergent in addition to the self-emulsifying component. In certain embodiments, the non-ionic detergent is selected from the group consisting of polysorbate-20, polysorbate-40, polysorbate-80, lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose, carboxymethyl cellulose, n-octylglucoside, n-dodecylglucoside, n-dodecylmaltoside, octanoyl-N-methylglucamide, decanoyl-N-methylglucamide, Triton™-X-100, Triton™-X-114, Thesit™, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), and N-dodecyl=N,N-dimethyl-3-ammonio-1-propane sulfonate. In other embodiments, one of the above-listed non-ionic detergents is selected.

In certain, more specific embodiments, a non-ionic detergent used in the described methods and compositions is a polysorbate-based detergent. Examples of polysorbate-based detergents are detergents derived by covalently bonding polyethoxylated sorbitan to a fatty acid. More specific embodiments of polysorbate-based detergents are polysorbate-20, polysorbate-40, and polysorbate-80.

For example, polysorbate 80 (Tween-80) is a non-ionic detergent derived from polyethoxylated sorbitan and oleic acid and having the following structure:

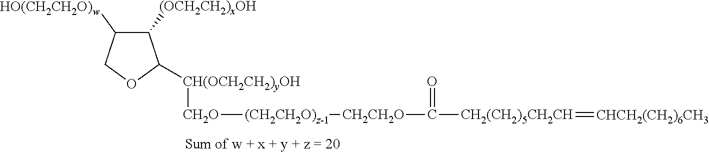

In the case of polysorbate 80, the moiety shown on the right side is a mixture of fatty acids, containing 60-70% oleic acid (as depicted), with the balance being primarily linoleic, palmitic, and stearic acids.

In a more specific embodiment, the polysorbate 80 constitutes 3-15% weight/weight inclusive of an oil-based liquid formulation used in the described methods and compositions. In a more specific embodiment, the percentage is 5-14% inclusive. In a more specific embodiment, the percentage is 7-12% inclusive. In more specific embodiments, the percentage is 10%, or alternatively 5%.

Chelators of Divalent Cations

The chelator of divalent cations utilized in the described methods and compositions is, in one embodiment, any physiologically acceptable compound having a high affinity for at least one of calcium, magnesium, and manganese ions. In another embodiment, the chelator is selected from the group consisting of citrate or a salt thereof; ethylenediamine tetracetic acid (EDTA) or a salt thereof (for example disodium EDTA and calcium disodium EDTA); EGTA (ethylene glycol tetraacetic acid) or a salt thereof; diethylene triamine pentaacetic acid (DTPA) or a salt thereof; and BAPTA (1,2-bis(o-aminophenoxy)ethane-N,N,N′,N′-tetraacetic acid) or a salt thereof. In other embodiments, one of the above-listed chelators is utilized. In more specific embodiments, the chelator is EDTA.

Oils

Those of skill in the art will appreciate, in light of the present findings, that various oils may be utilized as the basis of their liquid phase of the described compositions. In certain embodiments, the oil may be any physiologically acceptable oil that is liquid at ambient temperature.

In more specific embodiments, the oil comprises an omega-3 fatty acid. In other embodiments, the omega-3 fatty acid is an omega-3 polyunsaturated fatty acid. In another embodiment, the omega-3 fatty acid is DHA, an omega-3, polyunsaturated, 22-carbon fatty acid also referred to as 4, 7, 10, 13, 16, 19-docosahexaenoic acid. In another embodiment, the omega-3 fatty acid is --linolenic acid (9, 12, 15-octadecatrienoic acid). In another embodiment, the omega-3 fatty acid is stearidonic acid (6, 9, 12, 15-octadecatetraenoic acid). In another embodiment, the omega-3 fatty acid is eicosatrienoic acid (ETA; 11, 14, 17-eicosatrienoic acid). In another embodiment, the omega-3 fatty acid is eicsoatetraenoic acid (8, 11, 14, 17-eicosatetraenoic acid). In one embodiment, the omega-3 fatty acid is eicosapentaenoic acid (EPA; 5, 8, 11, 14, 17-eicosapentaenoic acid). In another embodiment, the omega-3 fatty acid is eicosahexaenoic acid (also referred to as 5, 7, 9, 11, 14, 17-eicosahexaenoic acid). In another embodiment, the omega-3 fatty acid is docosapentaenoic acid (DPA; 7, 10, 13, 16, 19-docosapenatenoic acid). In another embodiment, the omega-3 fatty acid is tetracosahexaenoic acid (6, 9, 12, 15, 18, 21-tetracosahexaenoic acid).

In other embodiments, the oil is a naturally-occurring oil comprising an omega-3 fatty acid. In more specific embodiments, the oil is selected from the group consisting of a fish oil, canola oil, flaxseed oil, algal oil and hemp seed oil. In more specific embodiments, the oil is a fish oil. Several types of fish oil have been tested in the described compositions and have all been found to work equally well.

In other embodiments, a liquid formulation utilized in the described method or composition is water-free. “Water-free” refers, in certain embodiments, to a formulation into which no aqueous components have been intentionally added. It does not preclude the presence of trace amounts of water that have been absorbed from the atmosphere into the components thereof. In another embodiment, the liquid formulation is free of aqueous components. In yet other embodiments, one or more oils are the only liquid components of the liquid formulation. In more specific embodiments, fish oil is the only liquid component of the liquid formulation.

Coatings

Those of skill in the art will appreciate, in light of the present findings, that various pH-sensitive coatings may be utilized in the described methods and compositions. In certain embodiments, any coating that inhibits digestion of the composition in the stomach of a subject may be utilized.

In other embodiments, the coating comprises a biodegradable polysaccharide. In other embodiments, a hydrogel is utilized. In other embodiments, the coating comprises one of the following excipients: chitosan, an aquacoat ECD coating, an azo-crosslinked polymer, cellulose acetate phthalate, cellulose acetate trimellitate (CAT), cellulose acetate butyrate, hydroxypropylmethyl cellulose phthalate, or poly vinyl acetate phthalate.

In other embodiments, a timed-release system such as Pulsincap™ is utilized.

In preferred embodiments, the described coated dosage forms release the core (containing the oil-based formulation) when pH reaches the range found in the intestines, which is alkaline relative to that in the stomach. In more specific embodiments, the coating comprises a pH-sensitive polymer. In various embodiments, either mono-layer or multi-layer coatings may be utilized.

In one embodiment, the coating is an enteric coating. Methods for enteric coating are well known in the art (see, for example, Siepmann F et al 2005). In more specific embodiments, a Eudragit™ coating is utilized as the enteric coating. Eudragit™ coatings are acrylic polymers, the use of which is well known in the art.

In another embodiment, microencapsulation is used as a stomach-resistant coating in the described compositions. Methods for microencapsulation are well known in the art.

In other embodiments, the coating is a capsule, of which gelatin capsules are a more specific embodiment. Methods for inserting an oil-based formulation into a gelatin capsule are well known in the art. In still other embodiments, the coating is a soft-gel, enteric-coated capsule.

In another embodiment, an oral pharmaceutical composition is provided, comprising an oil-based liquid formulation, wherein the oil-based liquid formulation comprises a therapeutic protein of up to 100 kilodaltons, a chelator of divalent cations, an isolated chymotrypsin/trypsin inhibitor, an isolated trypsin inhibitor, and a PEG ester of a fatty acid. In other embodiments, the liquid formulation has an anti-chymotrypsin activity of at least 50 mg. chymotrypsin inhibited per ml. of the liquid formulation. In other embodiments, the liquid formulation has both an anti-chymotrypsin activity of at least 50 mg. chymotrypsin inhibited per ml. of the liquid formulation and an anti-trypsin activity of at least 25 trypsin inhibited per ml. of the liquid formulation. In other embodiments, a free PEG is also present. In other embodiments, a non-ionic detergent is also present. In other embodiments, the liquid formulation consists essentially of a therapeutic protein of up to 100 kilodaltons, a chelator of divalent cations, a chymotrypsin/trypsin inhibitor, a trypsin inhibitor, and a PEG ester of a fatty acid. In other embodiments, the liquid formulation consists essentially of a therapeutic protein of up to 100 kilodaltons, a chelator of divalent cations, a chymotrypsin/trypsin inhibitor, a trypsin inhibitor, a PEG ester of a fatty acid, and a free PEG. In other embodiments, the liquid formulation consists essentially of a therapeutic protein of up to 100 kilodaltons, a chelator of divalent cations, a chymotrypsin/trypsin inhibitor, a trypsin inhibitor, a PEG ester of a fatty acid, a free PEG, and a non-ionic detergent.

Representative Formulations

Certain representative insulin formulations comprise insulin, Gelucire 44/14, EDTA, SBTI, and aprotinin in fish oil, coated in a capsule. Certain representative exenatide formulations contain exenatide, Gelucire 44/14, EDTA, SBTI, and aprotinin in fish oil, coated in a capsule.

In some embodiments, the invention provides a formulation having the following components: 8-20% Gelucire 44/14; 25-50 mg. per capsule rBBI, 25-50 mg isolated KTI; 100-200 mg EDTA; with a therapeutic protein, which may be 2-32 mg. per capsule insulin and/or 150-600 mcg. per capsule Exenatide, all combined into 0.5-0.7 ml. fish oil.

In some embodiments, the invention provides a formulation having the following components: 8-20% Gelucire 44/14; 12.5-25 mg. per capsule rBBI, 12.5-25 mg isolated KTI; 100-200 mg EDTA; with a therapeutic protein, which may be 2-32 mg. per capsule insulin and/or 150-600 mcg. per capsule Exenatide, all combined into 0.5-0.7 ml. fish oil.

In some embodiments, the invention provides a formulation having the following components: 12.5-25 mg. per capsule rBBI, 12.5-25 mg isolated KTI; 100-200 mg EDTA; with a therapeutic protein, which may be 2-32 mg. per capsule insulin and/or 150-600 mcg. per capsule Exenatide, all combined into 0.5-0.7 ml. fish oil.

In some embodiments, the invention provides a formulation having the following components: 25-50 mg. per capsule rBBI, 25-50 mg isolated KTI; 100-200 mg EDTA; with a therapeutic protein, which may be 2-32 mg. per capsule insulin and/or 150-600 mcg. per capsule Exenatide, all combined into 0.5-0.7 ml. fish oil.

In some embodiments, the invention provides a formulation having the following components: 50-100 mg. per capsule rBBI, 50-100 mg isolated KTI; 100-200 mg EDTA; with a therapeutic protein, which may be 2-32 mg. per capsule insulin and/or 150-600 mcg. per capsule Exenatide, all combined into 0.5-0.7 ml. fish oil.

In some embodiments, the invention provides a formulation having the following components: 75-125 mg. per capsule rBBI, 75-125 mg isolated KTI; 100-200 mg EDTA; with a therapeutic protein, which may be 2-32 mg. per capsule insulin and/or 150-600 mcg. per capsule Exenatide, all combined into 0.5-0.7 ml. fish oil.

In some embodiments, formulations containing recombinant BBI and insulin as described demonstrate a more rapid and qualitatively greater decline in circulating glucose levels, which effect is sustained for a longer period of time than that achieved with formulations comprising BBI chemically purified from Soy preparations.

In some embodiments, formulations containing recombinant BBI and a therapeutic protein- or peptide based therapeutic as described herein, provide a greater therapeutic window, in terms of sustained effect, for a longer period of time than that achieved with formulations comprising BBI chemically purified from Soy preparations, which may be evidenced in local effects post administration, or in some embodiments, circulating protein/peptide levels following administration.

In some embodiments, formulations containing recombinant BBI and a therapeutic protein- or peptide based therapeutic as described herein, provide a sustained effect, for an equal or longer period of time or provide equal or greater circulating levels of the protein/peptide following administration of the recombinant BBI, which recombinant BBI concentration is lower than the concentration of BBI chemically purified from Soy preparations, in an otherwise identical formulation.

According to this aspect and in some embodiments, the recombinant BBI concentration provided in a formulation of this invention will be at least 30% or in some embodiments, at least 35%, or in some embodiments, at least 40%, or in some embodiments, at least 45%, or in some embodiments, at least 50%, or in some embodiments, at least 55%, or in some embodiments, at least 60%, or in some embodiments, at least 65%, or in some embodiments, at least 70%, or in some embodiments, at least 75% less than the concentration of BBI chemically purified from Soy preparations used in an otherwise comparable or identical formulation.

It will be appreciated that reference to the term “otherwise comparable or identical” in regard to formulations of this invention contemplates identical key ingredients, which include the protein/peptide, BBI or KTI protease inhibitor, EDTA and/or fish oil or omega-3 fatty acid derived from fish oil, however other components of the formulation, may be comparable, as will be appreciated by the skilled artisan.

In one embodiment there is provided a formulation having the following components: 8-20% Gelucire 44/14; 50-100 mg. per capsule rBBI, or r BBI/isolated or recombinant KTI mixture; 20-30 mg. per capsule Aprotinin; and 100-200 mg EDTA; with a therapeutic protein, which may be 8-32 mg. per capsule insulin and/or 150-600 mcg. per capsule Exenatide, all combined into 0.5-0.7 ml. fish oil.

In another embodiment, there is provided a representative liquid insulin formulation containing insulin, Gelucire 44/14, EDTA, rBBI, KTI, and aprotinin in fish oil.

In other embodiments, the liquid formulation consists essentially of insulin, Gelucire 44/14, EDTA, rBBI, KTI, aprotinin, and fish oil.

In some embodiments, the formulations contain 8 mg insulin, 12% Gelucire 44/14, 150 mg EDTA, 75 mg total of rBBI and KTI, and 24 mg aprotinin in 0.5-0.7 ml. fish oil; or in some embodiments, 16 mg insulin, 12% Gelucire 44/14, 150 mg EDTA, 75 mg total of rBBI and KTI, and 24 mg aprotinin in 0.5-0.7 ml. fish oil; or in some embodiments, 16 mg insulin, 12% Gelucire 44/14, 150 mg EDTA, 150 mg total of rBBI and KTI, and 24 mg aprotinin in 0.5-0.7 ml. fish oil.

In some embodiments, the invention provides a formulation, wherein the ratio of the anti-trypsin activity to the anti-chymotrypsin activity of the composition is about 1.28:1. In some embodiments, the formulations containing the stated amount of rBBI alone is sufficient for protease inhibitor activity and no additional proteases are included in same.

In other embodiments, the above composition further comprises a non-ionic detergent. In more specific embodiments, the non-ionic detergent is a polysorbate-based detergent. In even more specific embodiments, the polysorbate-based detergent is polysorbate 80. Preferably, the polysorbate 80 constitutes 3-10% weight/weight inclusive of the oil-based liquid formulation.

The liquid formulation may be coated by a soft-gel, enteric-coated capsule. Specific formulations described in this paragraph also encompass, in certain embodiments, scaled-up and scaled-down formulation containing the same components in the same ratios.

Some representative oral exenatide formulations comprise exenatide, EDTA, rBBI, KTI, and aprotinin in fish oil. In other embodiments, the liquid formulation consists essentially of exenatide, Gelucire 44/14, EDTA, rBBI, KTI, aprotinin, and fish oil. More specific formulations contain 150 microgram (mcg) exenatide, 150 mg EDTA, 75 mg total of rBBI and KTI, and 24 mg aprotinin in 0.5-0.7 ml. fish oil; 300 mcg exenatide, 150 mg EDTA, 75 mg total of rBBI and KTI, and 24 mg aprotinin in 0.5-0.7 ml. fish oil; and 300 mcg exenatide, 150 mg EDTA, 150 mg total of rBBI and KTI, and 24 mg aprotinin in 0.5-0.7 ml. fish oil. In some embodiments, the formulations containing the stated amount of rBBI alone is sufficient for protease inhibitor activity and no additional proteases are included in same.

In a still more specific formulation, the ratio of the anti-trypsin activity to the anti-chymotrypsin activity of the composition is between 1.5:1 and 1:1, inclusive. In even more specific embodiments, the ratio is about 1.28:1. The liquid formulation may be coated by a soft-gel, enteric-coated capsule. In other embodiments, the above composition further comprises a non-ionic detergent. In more specific embodiments, the non-ionic detergent is a polysorbate-based detergent. In even more specific embodiments, the polysorbate-based detergent is polysorbate 80. Preferably, the polysorbate 80 constitutes 3-10% weight/weight inclusive of the oil-based liquid formulation. The liquid formulation may be coated by a soft-gel, enteric-coated capsule.

Therapeutic Indications

In another aspect is provided use of an rBBI described herein in the preparation of a medicament for orally administering an active ingredient to a subject.

In still another aspect is provided a method for making a pharmaceutical composition, comprising the steps of: (a) producing a mixture comprising an rBBI described herein and an active ingredient; and (b) formulating the mixture into a pharmaceutically acceptable formulation.

As referred to herein, the step of formulating comprises the steps of optionally adding excipients, milling, coating, and the like, as appropriate for the desired pharmaceutical composition. These steps are well within the ability of those skilled in the art.

In certain embodiments, an active ingredient as referred to herein is sensitive to degradation or inactivation in the human digestive tract.

In another aspect is provided an oral pharmaceutical composition described herein for orally administering an active ingredient to a subject. In certain preferred embodiments, the active ingredient is sensitive to degradation or inactivation in the human digestive tract. In more specific embodiments, the active ingredient may be a therapeutic protein. In other embodiments, the active ingredient is a non-protein molecule that is sensitive to degradation or inactivation in the human digestive tract.

In another aspect is provided use of an oral pharmaceutical composition described herein in the preparation of a medicament for orally administering a therapeutic protein to a subject.

In another aspect is provided a method for orally administering a therapeutic protein to a subject, the method comprising the step of administering to a subject an oral pharmaceutical composition described herein, thereby orally administering a therapeutic protein to a subject.

In another aspect is provided a pharmaceutical composition described herein for treating diabetes in a human, where, in some embodiments, the therapeutic protein is in various embodiments, insulin, exenatide, or a combination thereof.

In yet another aspect is provided a use of a combination of ingredients described herein in the preparation of a medicament for treating diabetes in a human, where, in some embodiments, the therapeutic protein is in various embodiments, insulin, exenatide, or a combination thereof.

In still another aspect is provided a method for treating diabetes, the method comprising the step of administering to a subject in need of such treatment a pharmaceutical composition described herein, where, in some embodiments, the therapeutic protein is in various embodiments insulin, exenatide, or a combination thereof, thereby treating diabetes. In certain embodiments, the subject is a human subject, while in other embodiments the subject is a non-human mammal.

In an additional aspect is provided a pharmaceutical composition described herein for treating unstable diabetes in a human. In another aspect, a pharmaceutical composition described herein is provided for treating an elevated fasting blood glucose level in a human.

In yet another aspect is provided a use of a combination of ingredients described herein in the preparation of a medicament for treating unstable diabetes in a human. Additionally, a use is provided of a combination of ingredients described herein in the preparation of a medicament for treating an elevated fasting blood glucose level in a human.

Additionally, there is provided a method for treating unstable diabetes, the method comprising the step of administering to a subject in need of such treatment a pharmaceutical composition described herein, thereby treating unstable diabetes. Further is provided a method for reducing an elevated fasting blood glucose level, the method comprising the step of administering to a subject in need of such treatment a pharmaceutical composition described herein, thereby reducing an elevated fasting blood glucose. In certain preferred embodiments, the subject is a human subject.

Unstable Diabetes

Physicians skilled in the art will appreciate that unstable diabetes, also known as glycemic lability, can be diagnosed by a number of acceptable standard procedures. One such procedure appears in Ryan et al, 2004. In this procedure, subjects are asked to monitor their glucose levels with a minimum of 2 capillary glucose readings a day. Patients record all measured glucose values and details about hypoglycemic occurrences over a 4-week period on sheets. On any occasion that glucose is recorded as <3.0 mmol/1, the subject is asked to describe the details of the event on the questionnaire, including which symptoms occur and whether outside help from a third party is obtained to either recognize or treat the hypoglycemic reaction. A reaction is considered severe if the individual had lost control of the situation and required outside help to treat the hypoglycemic event. Other such methods involve calculation of the MAGE index (Service et al 1970) or the “M value” (Schlichtkrull et al) or utilize continuous glucose monitoring systems (Kessler et al). Unstable diabetes is typically associated with elevated fasting blood glucose and/or hypoglycemic episodes.

It is to be understood that the invention contemplates oral compositions for treating a variety of diseases/disorders as described herein, including any form of diabetes, as will be appreciated by the skilled artisan.

In one embodiment, the terms “treating” or “treatment” includes preventive as well as disorder remittive treatment. The terms “reducing”, “suppressing” and “inhibiting” have their commonly understood meaning of lessening or decreasing, in another embodiment, or delaying, in another embodiment, or reducing, in another embodiment the incidence, severity or pathogenesis of a disease, disorder or condition. In embodiment, the term treatment refers to delayed progression of, prolonged remission of, reduced incidence of, or amelioration of symptoms associated with the disease, disorder or condition. In one embodiment, the terms “treating” “reducing”, “suppressing” or “inhibiting” refer to a reduction in morbidity, mortality, or a combination thereof, in association with the indicated disease, disorder or condition. In one embodiment, the term “progression” refers to an increasing in scope or severity, advancing, growing or becoming worse. The term “recurrence” means, in another embodiment, the return of a disease after a remission. In one embodiment, the methods of treatment of the invention reduce the severity of the disease, or in another embodiment, symptoms associated with the disease, or in another embodiment, reduces the number of biomarkers expressed during disease.

In one embodiment, the term “treating” and its included aspects, refers to the administration to a subject with the indicated disease, disorder or condition, or in some embodiments, to a subject predisposed to the indicated disease, disorder or condition. The term “predisposed to” is to be considered to refer, inter alia, to a genetic profile or familial relationship which is associated with a trend or statistical increase in incidence, severity, etc. of the indicated disease. In some embodiments, the term “predisposed to” is to be considered to refer, inter alia, to a lifestyle which is associated with increased risk of the indicated disease. In some embodiments, the term “predisposed to” is to be considered to refer, inter alia, to the presence of biomarkers which are associated with the indicated disease, for example, in cancer, the term “predisposed to” the cancer may comprise the presence of precancerous precursors for the indicated cancer.

In some embodiments, the term “reducing the pathogenesis” is to be understood to encompass reducing tissue damage, or organ damage associated with a particular disease, disorder or condition. In another embodiment, the term “reducing the pathogenesis” is to be understood to encompass reducing the incidence or severity of an associated disease, disorder or condition, with that in question. In another embodiment, the term “reducing the pathogenesis” is to be understood to encompass reducing the number of associated diseases, disorders or conditions with the indicated, or symptoms associated thereto.

Methods of Producing Pharmaceutical Compositions

Also provided herein are methods of producing pharmaceutical compositions. In certain embodiments, the method comprises the steps of optionally combining molten Gelucire (for example Gelucire 44/14) with fish oil, cooling the mixture, then adding, in powder form, EDTA, SBTI, aprotinin, and a therapeutic protein or peptide, for example insulin, or in other embodiments, exenatide, although those skilled in the art will appreciate in light of the present findings that other therapeutic proteins or peptides may be used as well. In other embodiments, the method comprises the steps of optionally combining molten Gelucire (for example Gelucire 44/14) with fish oil, cooling the mixture of, then adding, in powder form, EDTA, BBI, and a therapeutic protein or peptide. In certain embodiments, the powder components are added in the listed order. In other embodiments, the resulting mixture is optionally mixed and/or homogenized.

“Consisting essentially of”, as used herein, limits the scope of the invention to the specified materials or steps and those that do not materially affect the basic and novel characteristics of the claimed invention.

In one embodiment, the term “about” refers to a variance of from 1-10%, or in another embodiment, 5-15%, or in another embodiment, up to 10%, or in another embodiment, up to 25% variance from the indicated values, except where context indicates that the variance should not result in a value exceeding 100%.

In certain embodiments of the invention “weight” refers to “dry weight”. In other embodiments “weight” refers to total weight.

In some embodiments, the term “comprise” or grammatical forms thereof, refers to the inclusion of the indicated components of this invention, as well as inclusion of other active agents, and pharmaceutically acceptable carriers, excipients, emollients, stabilizers, etc., as are known in the pharmaceutical industry.

In one embodiment, the present invention provides combined preparations. In one embodiment, the term “a combined preparation” defines especially a “kit of parts” in the sense that the combination partners as defined above can be used independently or in different combinations i.e., simultaneously, concurrently, separately or sequentially.

In some embodiments, of the compositions of this invention will consist essentially of the components as herein described, in any form or embodiment as described herein. In some embodiments, the term “comprise” refers to the inclusion of the indicated components as herein described, such as the rBBI and therapeutic peptides or therapeutic proteins of this invention, as well as inclusion of other active agents, and pharmaceutically acceptable carriers, excipients, emollients, stabilizers, etc., as are known in the pharmaceutical industry. In some embodiments, the term “consisting essentially of” refers to a composition, whose only active ingredient is the indicated therapeutic peptides or therapeutic proteins of this invention and rBBI, however, other compounds may be included which are for stabilizing, preserving, etc. the formulation, but are not involved directly in the therapeutic effect of the indicated active ingredient and other protease inhibitors may be included, however, the inclusion of rBBI is known to be an essential component of the composition to promote prolonged, sustained activity of the therapeutic peptides or therapeutic proteins of this invention. In some embodiments, the term “consisting essentially of” refers to a composition, whose only active ingredient with a comparable mode of action, or comparable molecular target is the indicated active ingredient, however, other active ingredients may be incorporated, with such secondary active ingredients acting on different targets, or in a palliative capacity and same represent the therapeutic peptides or therapeutic proteins of this invention, and such composition will still further contain rBBI, as described. In some embodiments, the term “consisting essentially of” may refer to components which facilitate the release of the active ingredient, in addition to rBBI which serves to promote sustained delivery/preventing degradation, etc. of the therapeutic peptides or therapeutic proteins of this invention, as herein described. In some embodiments, the term “consisting” refers to a composition, which contains therapeutic peptides or therapeutic proteins of this invention as herein described as the only active ingredient, a rBBI and a pharmaceutically acceptable carrier or excipient.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

It will be understood by those skilled in the art that various changes in form and details may be made therein without departing from the spirit and scope of the invention as set forth in the appended claims. Those skilled in the art will recognize, or be able to ascertain using no more than routine experimentation, many equivalents to the specific embodiments of the invention described herein. Such equivalents are intended to be encompassed in the scope of the claims.

All publications, patents, and patent applications mentioned herein are hereby incorporated by reference in their entirety as if each individual publication or patent was specifically and individually indicated to be incorporated by reference. In case of a conflict between the specification and an incorporated reference, the specification shall control. Where number ranges are given in this document, endpoints are included within the range. Furthermore, it is to be understood that unless otherwise indicated or otherwise evident from the context and understanding of one of ordinary skill in the art, values that are expressed as ranges can assume any specific value or subrange within the stated ranges, optionally including or excluding either or both endpoints, in different embodiments of the invention, to the tenth of the unit of the lower limit of the range, unless the context clearly dictates otherwise. Where a percentage is recited in reference to a value that intrinsically has units that are whole numbers, any resulting fraction may be rounded to the nearest whole number.

In the claims articles such as “a,”, “an” and “the” mean one or more than one unless indicated to the contrary or otherwise evident from the context. Claims or descriptions that include “or” or “and/or” between members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides, in various embodiments, all variations, combinations, and permutations in which one or more limitations, elements, clauses, descriptive terms, etc., from one or more of the listed claims is introduced into another claim dependent on the same base claim unless otherwise indicated or unless it would be evident to one of ordinary skill in the art that a contradiction or inconsistency would arise. Where elements are presented as lists, e.g. in Markush group format or the like, it is to be understood that each subgroup of the elements is also disclosed, and any element(s) can be removed from the group.

It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in haec verba herein. Certain claims are presented in dependent form for the sake of convenience, but Applicant reserves the right to rewrite any dependent claim in independent format to include the elements or limitations of the independent claim and any other claim(s) on which such claim depends, and such rewritten claim is to be considered equivalent in all respects to the dependent claim in whatever form it is in (either amended or unamended) prior to being rewritten in independent format.

The following examples describe certain embodiments of the invention and should not be construed as limiting the scope of what is encompassed by the invention in any way.

### EXPERIMENTAL DETAILS SECTION

### Example 1: Production of Recombinant BBI

### Materials and Methods

The gene for the expression of recombinant Bowman-Birk type proteinase inhibitor (rBBI) was codon-optimized and synthesized in a Pichia pastoris system. The plasmid used for secretary expression of BBI was the pPIC9K Pichia expression vector (Invitrogen) expressed in the host: Pichia Pastoris GS115. The biased and optimized BBI DNA sequence was cloned in frame with the Saccharomyces cerevisiae α-mating factor pre-sequence (STE13 cleavage site deleted) in the pPIC9K plasmid to obtain the clone that will express secreted BBI.

The Pichia pastoris host optimized nucleic acid sequence for BBI is as follows:

[SEQ ID NO: 1]
GATGACGAAAGCTCTAAGCCATGCTGCGATCAGTGTGCTTGT
ACCAAGTCCAACCCACCACAGTGTAGATGCTCCGACATGCGT
TTGAACTCCTGTCACTCC GCTTGCAAGTCCTGTATCTGTGC
CTTGTCCTACCCAGCTCAGTGTTTCTGCGTCGACATCACCGA
CTTCTGCTACGAGCCATGTAAGCCATCTGAGGACGATAAGGA
AAAC [symbol] .

The amino acid sequence of BBI isolated from Glycine max (Soybean) (Glycine hispida) of UniProtKB—P01055 (IBB1_SOYBN) (110aa) is as follows:

[SEQ ID NO: 2]
MVVLKVCLVLLFLVGGTTSANLRLSKLGLLMKSDHQHSNDD
ESSKPCCDQCACTKSNPPQCRCSDMRLNSCHSACKSCICAL
SYPAQCFCVDITDFCYEPCKPSEDDKEN.

The bold represents the start codon, underlined represents the signal peptide and italicized portion represents the pro-peptide. Residue designations in regular font correspond to the E00124 chain, the sequence of which was subjected to gene biasing and optimization for expression in Pichia, and In silico translation of the optimized gene sequence yielded the identical sequence to that of SEQ ID NO: 2.

Two stop codons were added to the 3′ end of the final DNA sequence (italicized, bolded and underlined text in SEQ ID NO: 1). The biased and optimized BBI DNA sequence was cloned in frame with the Saccharomyces cerevisiae α-mating factor pre-sequence (STE13 cleavage site deleted) in the pPIC9K plasmid to obtain the clone that will express secreted BBI. The recombinant constructs were then re-characterized by sequencing.

The expression of the recombinant product was accomplished via standard methodology. Briefly, Pichia was cultured in YPD media at 30° C. with shaking to an OD₆₀₀ of 0.8 to 1.0, after which cells were harvested, washed and suspended and washed in 100 mM LiCl and then transformed. A standard transformation protocol was used. Briefly, the LiCl-cell solution was then washed and resuspended in a 50% PEG-4000 solution containing 1M LiCl single-stranded fragmented salmon sperm DNA and plasmid DNA in sterile water (5-10 μg; 100-200 ng/μl). Cells were incubated at 30° C. for 30 minutes then heat shocked at 42° C. for 20-25 minutes, following which cells were washed and resuspended in YPD medium, incubated at 30° C. then plated on YNB-His selective plates and incubated for a further 2-3 days at 30° C.

Around 1000 putative clones were screened to obtain a high-yielding clone. Around 5-6 clones were scaled up for 200 ml, 1 L and 5 L Fermenter batches for process optimization. The purified secreted rBBI protein from Clone B17 was subjected to N- and C-terminus sequencing by MALDI-TOF-MS with the observed N- and C-terminal sequence being the same as expected. Spectrum data confirms the observed mass is same as expected mass with 99% homogeneity. Thus the processing of rBBI from Clone B17 was intact without any deletion or addition of extra residues from signal peptide. Clone B17 was therefore selected as a potential high yielding clone and was further taken for scale up.

### Example 2: Characterization of the Recombinant BBI Product

The B17 clone was further studied for expression, purification and material generation at 200 ml, 5 L and 50 L scale and purified protein was characterized using different analytical methods.

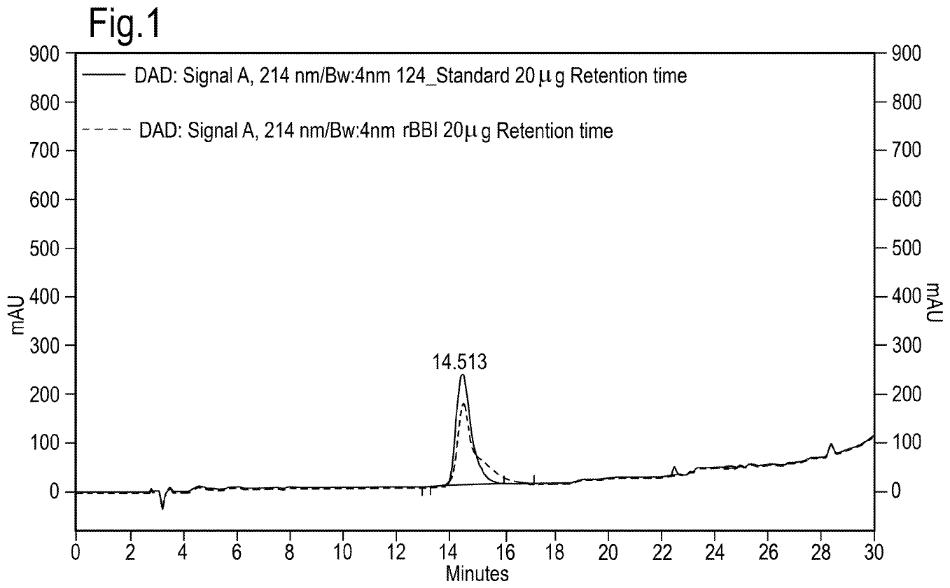

RP-HPLC analysis was conducted by standard methodology and the results are depicted in FIG. 1, depicting an RP-HPLC analysis and overlay with available standard. The final purified rBBI showed same retention times as observed with standard purified BBI.

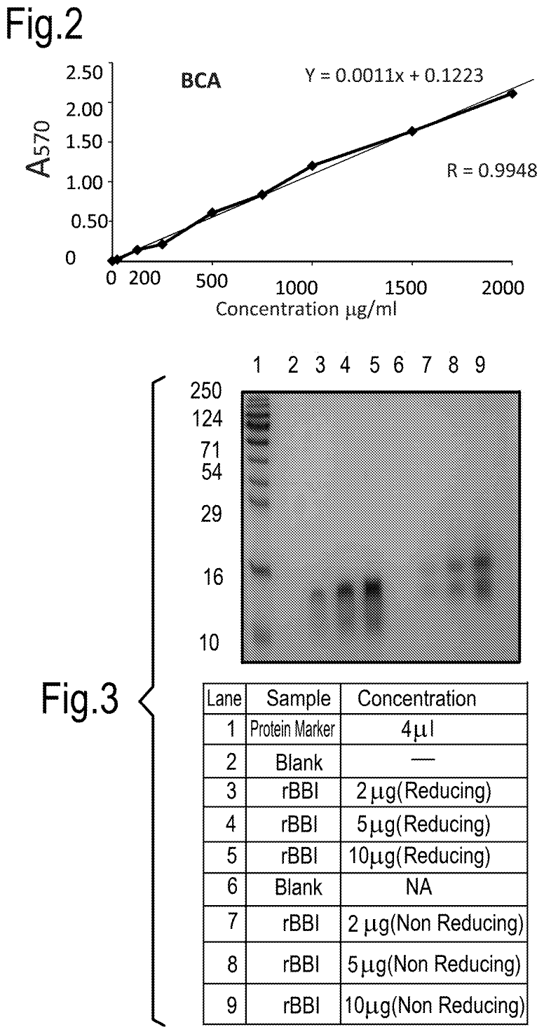

A lyophilized powder was prepared from the purified rBBI and BCA quantification of total protein content was assessed using standard methodology (FIG. 2) and SDS-PAGE analysis of a 15% gel under reducing and non-reducing conditions and stained with Coomassie blue verified the protein size (FIG. 3).

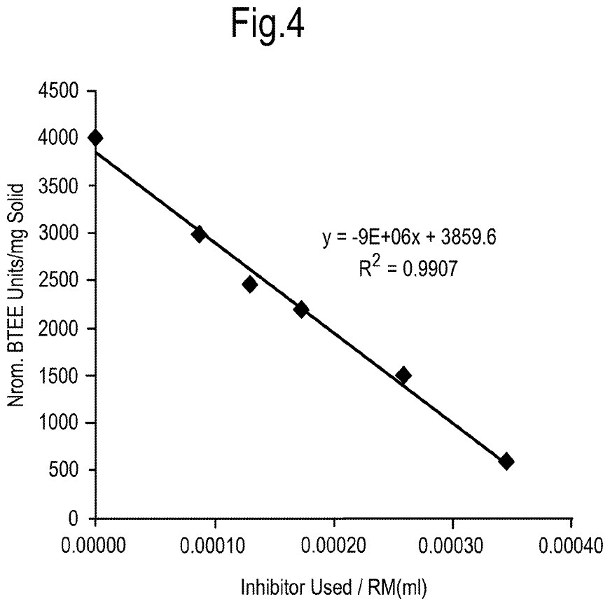

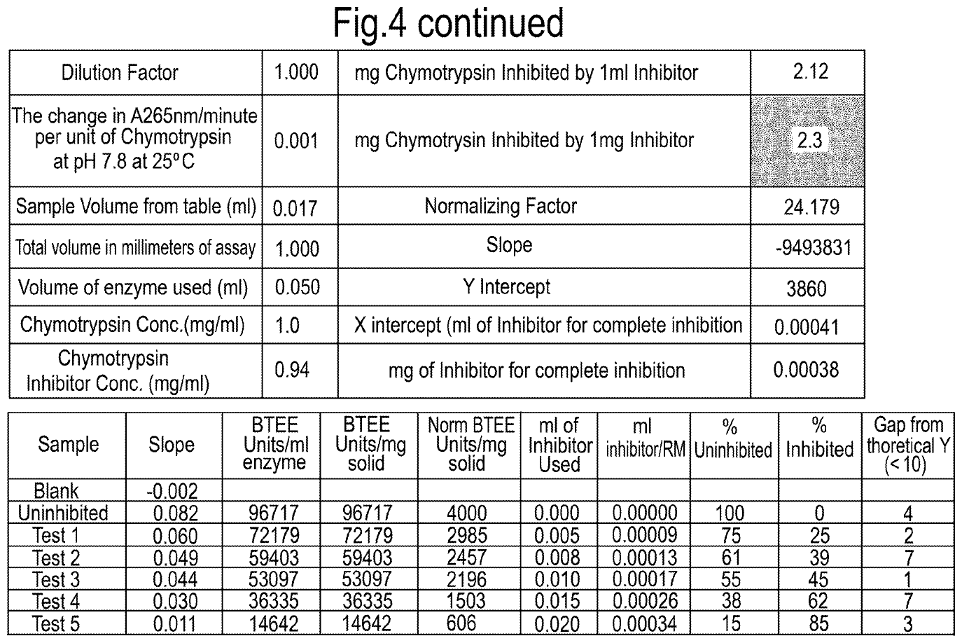

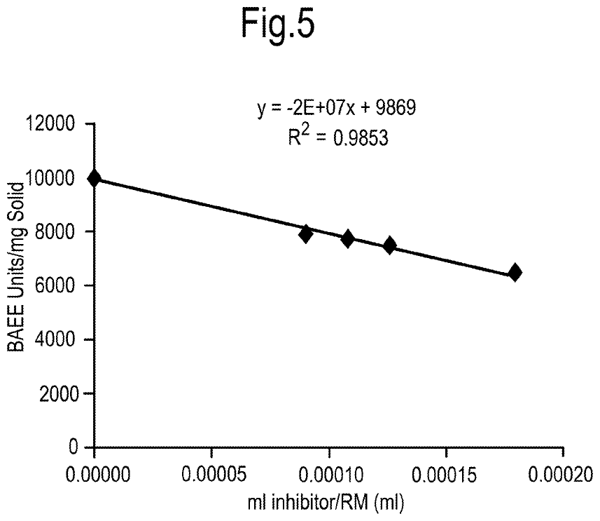

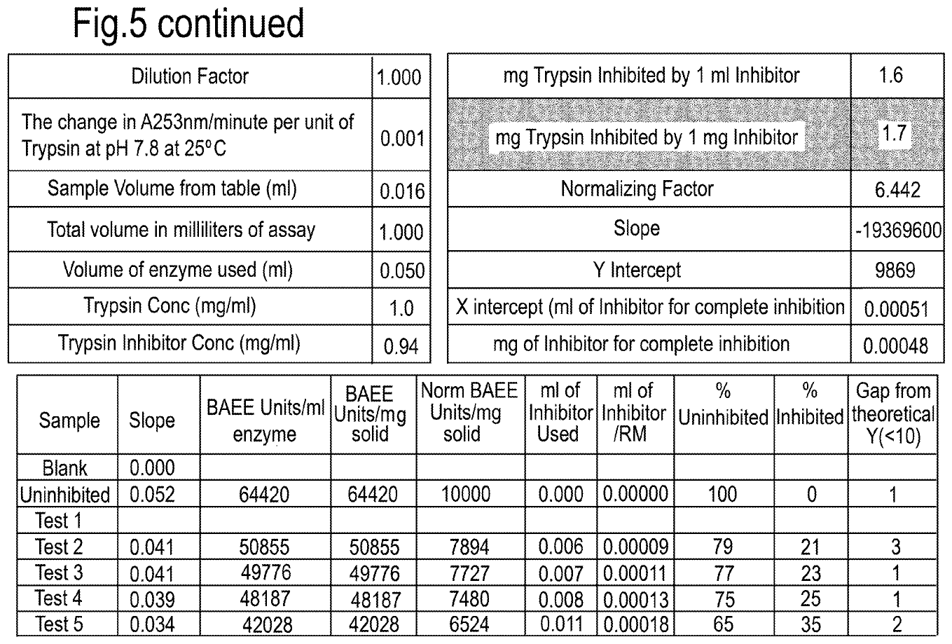

Protease inhibitor enzymatic activity was verified for the recombinant product, and demonstrable inhibition of chymotrypsin activity was achieved (FIG. 4) as well as inhibition of trypsin activity (FIG. 5). Surprisingly, the results of these assays demonstrated far greater inhibition of enzymatic activity in use of the recombinant protein as compared to purified SBTI purified from soya.

### Example 3: Recombinant Protease Inhibitor Provides for Surprising

### Efficacy

### Materials and Methods

Fasting 3-4 month old, 25-30 kg in weight non-SPF commercially purchased pigs (obtained from Ibelin Farm (Israel) (n=8) were anesthetized with isoflurane (2 L O₂ per minute and 3-5% isoflurane), tagged for ease of identification and intubated. Access to water was ad libitum. Animals were positioned on their left side and formulations were administered through an endoscopic catheter, under endoscopic guidance, directly into the duodenum. This study was conducted following approval for same by the Israeli Council of Animal Experimentation, Ministry of Health (protocol no. IL-16-11-400).

After injection of 1 ml test formulation, 150-180 ml of air was injected, to ensure delivery of the formulation to the duodenum. Blood was drawn from a central vein catheter (that was inserted on the first day of the experiments and was replaced as needed) at the following time points: 15 min and right before dosing, and every 15 min for up to 3 hour postdosing and then 3:20, 3:40, 4:00 and 4:30 post-dosing. A small portion (5 μl) of the blood samples was used for glucose concentration determination with a glucometer. When readings were <10 mg/dL, samples were tested again and the average value of the two readings is presented. A minimum two-day washout period was enforced between treatments.

Test conditions were conducted with 150 mg EDTA, 8 mg insulin and either purified recombinant BBI produced as described herein above, or as purified from SBTI extracted from soybean flour as described in PCT International Application Publication Number WO/2013/114369.

The test conditions were as described in Table 1 below, with each test item being tested in 4 different pigs:

| | Insulin | EDTA | KTI | rBBI | SBTI |
|---|---|---|---|---|---|
| Item ID | (mg) | (mg) | (mg) | (mg) | (mg) |
| Test item 1 | 8 | 150 | 0 | 0 | 100 |
| Test item 2 | 8 | 150 | 0 | 100 | 0 |
| Test item 3 | 8 | 150 | 50 | 50 | 0 |
| Test item 4 | 8 | 150 | 25 | 100 | |

All test items were prepared in 1 mL fish oil and stored refrigerated. Prior to use, samples were brought to room temperature and thoroughly mixed before administration.

Data analysis included assessment of circulating glucose levels as a function of time.

Animals were observed for the duration of the study and no abnormal clinical observations were observed during this study, with animal weights remaining stable throughout the study.

Results:

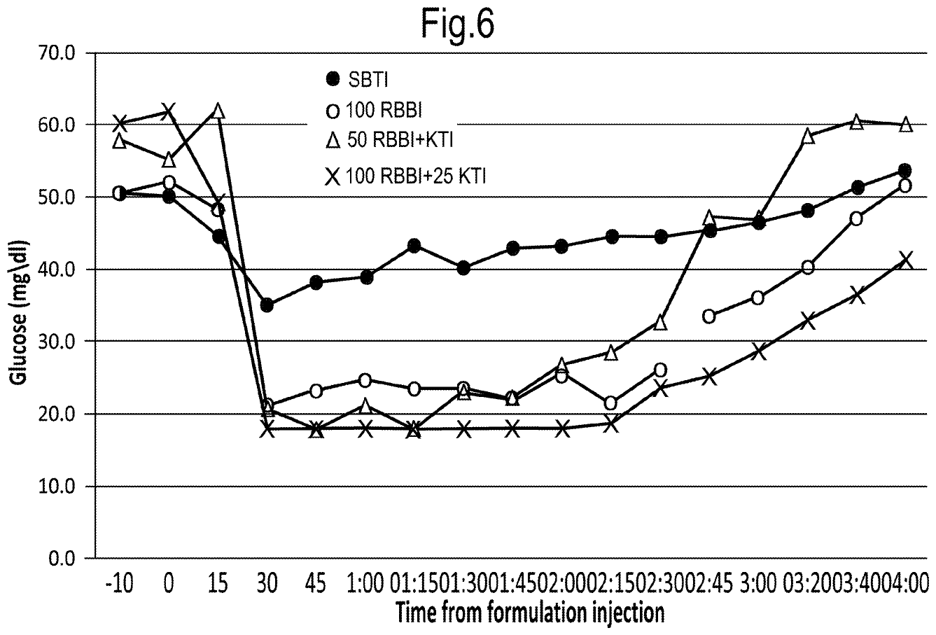

While all formulations produced a decline in circulating glucose levels (FIG. 6), use of the recombinant BBI surprisingly consistently led to lower glucose levels for a sustained period of time.

As has been shown previously, SBTI containing formulations provided a reduction in circulating glucose levels from 50 mg/dl to roughly 35 mg/dl within 30 minutes, but already by less than three hours post administration circulating levels returned essentially to baseline levels and moreover, the return to baseline levels was incremental, beginning as early as one hour after receipt of the oral dosage.

Quite surprisingly, all groups provided recombinant BBI demonstrated a more rapid and qualitatively greater decline in circulating levels to about 20 mg/dl and this effect was sustained for at least 2 hours after dosing, after which an incremental increase was evident, as well, however, animals given 100 mg recombinant BBI (rBBI) with 25 mg KTI by 4 hours out still had not returned to baseline, and animals give 100 mg rBBI alone did not return to baseline until more than 3.5 hours elapsed post-administration. Surprisingly, animals administered only 50 mg rBBI and KTI did not return to baseline, as well, until more than 3 hours had elapsed post administration.

Thus the results supported that surprisingly, rBBI was more effective in lowering circulating glucose levels as compared to purified BBI containing compositions and that the superior effect potentially persisted over a long period

### Example 4: Recombinant Protease Inhibitor Provides for Surprising Efficacy Over Time

### Materials and Methods

Fasting 3-4 month old, 25-30 kg in weight non-SPF commercially purchased pigs (obtained from Ibelin Farm (Israel) (n=8) were anesthetized with isoflurane (2 L O₂ per minute and 3-5% isoflurane), tagged for ease of identification and intubated. Access to water was ad libitum. Animals were positioned on their left side and formulations were administered through an endoscopic catheter, under endoscopic guidance, directly into the duodenum. This study was conducted following approval for same by the Israeli Council of Animal Experimentation, Ministry of Health (protocol no. IL-16-11-400).

After injection of 1 ml test formulation, 150-180 ml of air were injected, to ensure delivery of the formulation to the duodenum. Blood was drawn from a central vein catheter (that was inserted on the first day of the experiments and was replaced as needed) at the following time points: 15 min and right before dosing, and every 15 min for up to 3 hour post-dosing and then 3:20, 3:40, 4:00, 4:30 and 5 hours post-dosing. A small portion (5 μl) of the blood samples was used for glucose concentration determination with a glucometer. When readings were <10 mg/dL, samples were tested again and the average value of the two readings is presented. A minimum two-day washout period was enforced between treatments.

Test conditions were conducted with 150 mg EDTA, and 4 mg insulin and increasing quantities of recombinant BBI produced as described herein above.

The test conditions were as described in Table 2 below, with each test item being tested in 8-10 sessions in 7-8 different pigs:

TABLE 2
Treatment Protocol

| Item ID | Insulin (mg) | EDTA (mg) | rBBI (mg) |
|---|---|---|---|
| Test item 1 | 4 | 150 | — |
| Test item 2 | 4 | 150 | 12.5 |
| Test item 3 | 4 | 150 | 25 |
| Test item 4 | 4 | 150 | 50 |
| Test item 5 | 4 | 150 | 75 |

All test items were prepared in 1 mL fish oil and stored refrigerated. Prior to use, samples were brought to room temperature and thoroughly mixed before administration.

Data analysis included assessment of a change from baseline and percent change from baseline glucose area under the curve (AUC) were determined for each treatment session. In addition, the time from first drop to first rise in glucose readings was determined for each treatment session, as well.

Animals were observed for the duration of the study and no abnormal clinical observations were observed during this study, with animal weights remaining stable throughout the study.

### Results:

Example 3 demonstrated that the rBBI provided for enhanced glycemic control in that there was a greater decline in circulating glucose levels in subjects administered 8 mg insulin orally, when the protease inhibitor was a recombinant BBI, as opposed to that chemically purified. To further elaborate on the effects of rBBI, and to extend the results, it was of interest to optimize rBBI and evaluate the effects over time. Toward this end, oral formulations containing only 4 mg insulin, but containing 12.5, 25, 50 and 75 mg rBBI were similarly evaluated. Table 3 provides the results of these studies:

TABLE 3
rBBI glycemic control

| | Treatment Group Evaluated | | | | |
|---|---|---|---|---|---|
| | | 4 mg | 4 mg | 4 mg | 4 mg |
| | 4 mg | Insulin + | Insulin + | Insulin + | Insulin + |
| | Insulin + | EDTA + | EDTA + | EDTA + | EDTA + |
| Parameter | EDTA | 12.5 mg | 25 | 50 | 75 |
| Assessed: | alone | rBBI | mg rBBI | mg rBBI | mg rBBI |
| Average | 25935 | 24237 | 20864 | 24053 | 22483 |
| AUC ratio | | | | | |
| Average AUC | −2201 | −3005 | −6369 | −3029 | −4640 |
| ratio as value | | | | | |
| compared | | | | | |
| to baseline | | | | | |
| AUC change | 0 | −431 | −4989 | −2314 | −2960 |
| as compared to | | | | | |
| control | | | | | |
| Time in minutes | 80 | 113 | 140 | 121 | 100 |
| from initial | | | | | |
| decline in | | | | | |
| glucose levels to | | | | | |
| increasing | | | | | |
| glucose levels | | | | | |
| Time from initial | 100% | 142% | 175% | 152% | 126% |
| decline in | | | | | |
| glucose levels | | | | | |
| to increasing | | | | | |
| glucose levels | | | | | |
| expressed as | | | | | |
| percent | | | | | |

The lowest reduction from baseline glucose ratio AUCs following treatment with ORMD-0801 formulated with 25 mg rBBI, followed by the formulation containing 75 mg rBBI. When considering animal responses to treatment with formulations containing the protease inhibitor versus the control treatment containing no protease inhibitor, again, the formulations containing 25 mg rBBI and then 75 mg rBBI yielded the largest change from baseline AUC (−6,369 and −4,640, respectively) and correspondingly the largest change from control session AUC calculations (−4,989 and −2,960, respectively).

In terms of the sustainability of the effect, quite unexpectedly, rBBI containing formulations provided for a prolonged effect, with a mean 140 minute stretch between the first sign of glucose decline to first sign of its return toward baseline values in the tested subjects. This effect was 75% longer than the duration of the effect measured in the control animals

Thus, surprisingly and unexpectedly, although downstream purification steps of the BBI from purely chemical extraction means or chemical purification of recombinantly expressed proteins are essentially identical, when the source material for the BBI is rBBI, greater more sustained inhibitor activity was found in these studies.

Thus, quite unexpectedly, the recombinant BBI was sufficient, as a single protease inhibitor, to produce sustainable and qualitatively greater glycemic control.

1-77. (canceled)

78. An oral pharmaceutical composition comprising a therapeutic peptide or therapeutic protein of up to 100 kilodaltons, a chelator of divalent cations, and an isolated recombinantly expressed Bowman-Birk inhibitor (BBI);
wherein the recombinantly expressed BBI is expressed in a yeast expression system; and
wherein the yeast is Pichia pastoris.

79. The oral pharmaceutical composition of claim 78, wherein the recombinantly expressed BBI has a nucleotide sequence having at least 95% identity to SEQ ID NO:1.

80. The oral pharmaceutical composition of claim 79, wherein the recombinantly expressed BBI has a nucleotide sequence of SEQ ID NO:1.

81. The oral pharmaceutical composition of claim 78, further comprising a trypsin inhibitor other than a BBI.

82. The oral pharmaceutical composition of claim 78, wherein the therapeutic peptide or therapeutic protein is useful in treating a subject with a metabolic disease or disorder.

83. The oral pharmaceutical composition of claim 78, wherein the therapeutic peptide or therapeutic protein is insulin, influenza hemagglutinin, influenza neuraminidase, a glucagon, interferon gamma, interferon beta, interferon alpha, growth hormone, erythropoietin, GLP-1, a GLP-1 analogue, leptin, granulocyte colony stimulating factor (G-CSF), renin, growth hormone releasing factor, parathyroid hormone, thyroid stimulating hormone, follicle stimulating hormone, calcitonin, luteinizing hormone, glucagon, a clotting factor, an anti-clotting factor, atrial natriuretic factor, surfactant protein A (SP-A), surfactant protein B (SP-B), surfactant protein C (SP-C), surfactant protein D (SP-D), a plasminogen activator, bombesin, hematopoietic growth factor (colony-stimulating factor, multiple), a tumor necrosis factor (TNF) protein, enkephalinase, RANTES (regulated on activation normally T-cell expressed and secreted), human macrophage inflammatory protein (MIP-1-alpha), serum albumin, Mullerian-inhibiting substance, relaxin, mouse gonadotropin-releasing hormone, DNase, inhibin, activin, vascular endothelial growth factor (VEGF), a neurotrophic factor, neurotrophin-3,-4,-5, or -6 (NT-3, NT-4, NT-5, or NT-6), nerve growth factor, platelet-derived growth factor (PDGF), a fibroblast growth factor, a nerve growth factor, a transforming growth factor (TGF), insulin-like growth factor-I and -II (IGF-I and IGF-II), des (1-3)-IGF-I (brain IGF-I), insulin-like growth factor binding protein 1 (IGFBP-1), IGFBP-2, IGFBP-3, IGFBP-4, IGFBP-5, IGFBP-6, a keratinocyte growth factor, an osteoinductive factor, bone morphogenetic protein (BMP)-2, BMP-4, BMP-7, a colony stimulating factor (CSF), an interleukin (IL), superoxide dismutase, decay accelerating factor, a chemokine family member, or a complement factor.

84. The oral pharmaceutical composition of claim 83, wherein the therapeutic peptide or therapeutic protein is insulin or a GLP-1 analogue.

85. The oral pharmaceutical composition of claim 78, wherein the therapeutic peptide or therapeutic protein is an immunomodulatory compound.

86. The oral pharmaceutical composition of claim 85, wherein the immunomodulatory compound is glatiramer acetate.

87. The oral pharmaceutical composition of claim 78, wherein the chelator of divalent cations is ethylenediaminetetraacetic acid (EDTA).

88. The oral pharmaceutical composition of claim 78, wherein the oral pharmaceutical composition is an oil-based liquid formulation.

89. The oral pharmaceutical composition of claim 88, further comprising a polyethylene glycol (PEG) ester of a monoglyceride, a diglyceride, a triglyceride, or a mixture thereof.

90. The oral pharmaceutical composition of claim 78, further comprising gelatin and glycerol.

91. The oral pharmaceutical composition of claim 88, further comprising a non-ionic detergent.

92. The oral pharmaceutical composition of claim 88, wherein the oil is fish oil.

93. The oral pharmaceutical composition of claim 78, further comprising a coating that resists degradation in the stomach.

94. The oral pharmaceutical composition of claim 78, wherein the oral pharmaceutical composition promotes sustained activity of orally administered therapeutic peptides or therapeutic proteins in a subject, or wherein the oral pharmaceutical composition promotes sustained bioavailability of orally administered therapeutic peptides or therapeutic proteins in a subject.

95. The oral pharmaceutical composition of claim 94, wherein the sustained activity or the sustained bioavailability of orally administered therapeutic peptides or therapeutic proteins exceeds that of an otherwise identical oral pharmaceutical composition comprising a chemically purified BBI instead of the isolated recombinantly expressed BBI.

96. The oral pharmaceutical composition of claim 78, wherein the oral pharmaceutical composition provides a therapeutically effective amount of the peptide or protein, and wherein the BBI, or the therapeutic peptide or therapeutic protein of up to 100 kilodaltons, or a combination thereof is provided in the oral pharmaceutical composition at a lower concentration than would be therapeutically effective in an otherwise identical oral pharmaceutical composition comprising a chemically purified BBI instead of the isolated recombinantly expressed BBI.

97. A method for orally administering a therapeutic peptide or therapeutic protein to a subject in need thereof, comprising administering the oral pharmaceutical composition of claim 78 to the subject.

98. A method of preventing degradation of orally administered therapeutic peptides or therapeutic proteins in a subject in need thereof, comprising administering the oral pharmaceutical composition of claim 78 to the subject.

99. A method of promoting sustained activity of orally administered therapeutic peptides or therapeutic proteins in a subject in need thereof, comprising administering the oral pharmaceutical composition of claim 78 to the subject.

100. A method of promoting sustained bioavailability of orally administered therapeutic peptides or therapeutic proteins in a subject in need thereof, comprising administering the oral pharmaceutical composition of claim 78 to the subject.

101. A method of providing a therapeutically effective amount of a peptide or protein in an oral pharmaceutical composition to a subject in need thereof, comprising providing the oral pharmaceutical composition of claim 78;
wherein the BBI, or the therapeutic peptide or therapeutic protein of up to 100 kilodaltons, or a combination thereof, is provided at a lower concentration than would be therapeutically effective in an otherwise identical oral pharmaceutical composition comprising a chemically purified BBI instead of the isolated recombinantly expressed BBI and having a comparable therapeutic effect in the subject.
